SEQUENCE LISTING (16 sequences)

SEQ ID NO: 1 - DNA, 22 bp, unidentified
  source 1..22: mol_type = other DNA
gggaacagatgggcagaaatcg

SEQ ID NO: 2 - DNA, 22 bp, unidentified
  source 1..22: mol_type = other DNA
gctggcttttgaagactcctgc

SEQ ID NO: 3 - DNA, 24 bp, unidentified
  source 1..24: mol_type = other DNA
catgttcgtcatgggtgtgaacca

SEQ ID NO: 4 - DNA, 24 bp, unidentified
  source 1..24: mol_type = other DNA
atggcatggactgtggtcatgagt

SEQ ID NO: 5 - DNA, 43 bp, unidentified
  source 1..43: mol_type = other DNA
gactcatacagctggcactcgagggacgctggtcatgtgagac

SEQ ID NO: 6 - DNA, 39 bp, unidentified
  source 1..39: mol_type = other DNA
gactcatacagcggccgcaaagtgccttgagtctgctcc

SEQ ID NO: 7 - DNA, 52 bp, unidentified
  source 1..52: mol_type = other DNA
ttttattcagtaatttaattttgtaaaaacgttttttgctgctatggtctta

SEQ ID NO: 8 - DNA, 52 bp, unidentified
  source 1..52: mol_type = other DNA
taagaccatagcagcaaaaaacgtttttacaaaattaaattactgaataaaa

SEQ ID NO: 9 - DNA, 48 bp, unidentified
  source 1..48: mol_type = other DNA
tattgtggtttttttgttattgttgcatgcgttgcacttcttttttgg

SEQ ID NO: 10 - DNA, 47 bp, unidentified
  source 1..47: mol_type = other DNA
ccaaaaaagaagtgcaacgcatgcaacaataacaaaaaaccacaata

SEQ ID NO: 11 - DNA, 43 bp, unidentified
  source 1..43: mol_type = other DNA
actcatacagctggcactcgagatcccagacagtggatatgac

SEQ ID NO: 12 - DNA, 37 bp, unidentified
  source 1..37: mol_type = other DNA
actcatacagcggccgcattctcaccttgagaatgca

SEQ ID NO: 13 - DNA, 49 bp, unidentified
  source 1..49: mol_type = other DNA
cagatgtgcaataatttgtacaatgtatgccttaagcagaacaaatgtg

SEQ ID NO: 14 - DNA, 49 bp, unidentified
  source 1..49: mol_type = other DNA
cacatttgttctgcttaaggcatacattgtacaaattattgcacatctg

SEQ ID NO: 15 - protein, 11 aa, synthetic construct
KLWVLPKGGGC

SEQ ID NO: 16 - RNA, 23 nt, synthetic construct
  source 1..23: mol_type = other RNA
ttcatttggtataaaccgcgatt

(19) United States
(12) Patent Application Publication
(10) Pub. No.: US 2026/0000619 A1
(43) Pub. Date: Jan. 1, 2026

(54) BIOMIMETIC TORPEDO FOR STENT-FREE AND TARGETED GENE THERAPY TO PREVENT RESTENOSIS

(71) Applicant: University of Virginia Patent Foundation, Charlottesville, VA (US)

(72) Inventors: Kenneth Craig KENT, Charlottesville, VA (US); Lian-Wang GUO, Madison, WI (US); Xiujie XIE, Crozet, VA (US); Takuro SHIRASU, Charlottesville, VA (US); Bowen WANG, Charlottesville, VA (US)

(21) Appl. No.: 18/881,541

(22) PCT Filed: Jul. 6, 2023
(86) PCT No.: PCT/US2023/069688

§ 371 Date: Jan. 6, 2025

Related U.S. Application Data
(60) Provisional application No. 63/367,750, filed on Jul. 6, 2022.

(51) Int. Cl. A61K 9/48; A61K 31/7105; A61K 31/713; A61K 48/00; A61P 7/02

(52) U.S. Cl. CPC A61K 9/4816; A61K 31/7105; A61K 31/713; A61K 48/0041; A61P 7/02

(57) ABSTRACT

Disclosed herein is a biomimetic torpedo that can circumvent existing hurdles for RNA therapies. That is, RNA therapeutics can be harbored inside a torpedo shell made of neutrophil membranes in hybrid with a liposome membrane, which enables lesion targeting and shielding from immunogenicity. Further disclosed herein is a biomimetic targeting system that involves a neutrophil cell membrane capsule that encapsulates a therapeutic RNA.

### CROSS-REFERENCE TO RELATED APPLICATIONS

This application claims benefit of U.S. Provisional Application No. 63/367,750, filed Jul. 6, 2022, which is hereby incorporated herein by reference in its entirety.

### STATEMENT REGARDING FEDERALLY SPONSORED RESEARCH OR DEVELOPMENT

This invention was made with Government Support under Grant Nos. HL143469, HL29785, and HL133665 awarded by the National Institutes of Health. The Government has certain rights in the invention.

### SEQUENCE LISTING

This application contains a sequence listing filed in ST.26 format entitled “222117_2200_Sequence_Listing” created on Jun. 29, 2023, having 15,304 bytes. The content of the sequence listing is incorporated herein in its entirety.

### BACKGROUND

Stenotic cardiovascular diseases, and in particular, atherosclerosis, are the leading cause of morbidity and mortality worldwide despite improvements in medical therapies (Virani S S, et al. Circulation. 2021 143:e254-e743). Currently, two major clinical approaches are used to resume obstructed blood flow: angioplasty and bypass surgery (Zenati M A, et al. N Engl J Med. 2019 380:132-141; Lam A, et al. Vascular. 2020 28:747-755). Unfortunately, following either procedure, neointimal hyperplasia (IH) develops in the vessel wall, and if untreated, could narrow the vessel lumen and contribute to recurrent stenosis or bypass graft failure. Drug-eluting stents have been advanced to treat post-angioplasty IH but in-stent restenosis still occurs. Moreover, the drugs used on the stents (e.g. paclitaxel), which are indiscriminately anti-proliferative, are associated with heightened risks of thrombosis and mortality, as increasingly reported (Luscher T F, et al. Circulation. 2007 115:1051-8; Inoue T, et al. JACC Cardiovasc Interv. 2011 4:1057-66; Katsikis A and Serruys P W. J Thorac Dis. 2017 9:2296-2300). To date, there has been a lack of approved therapeutics to prevent bypass grafts from developing IH and subsequent post-surgical graft failure (Gregory E K, et al. J Control Release. 2018 274:69-80; Decano J L, et al. Circulation. 2021 143:2454-2470; Zhao C, et al. J Am Heart Assoc. 2020 9:e0184). Undoubtedly, postoperative IH, whether associated with angioplasty or bypass surgery, remains a major medical problem, and better/specific therapeutic agents for IH prevention are critically needed.

Vascular smooth muscle cells (SMCs) constitute major cellular populations and crucial functions in the normal vessel wall. However, when exposed to stimulants in a disturbed microenvironment, such as cytokines that surge in the injured vascular wall, SMCs undergo contractile-to-synthetic phenotypic switching, becoming proliferative, migratory, and de-differentiated (or further transdifferentiated to other cell types) (Chakraborty R, et al. JVS Vasc Sci. 2021 2:79-94; Alencar G F, et al. Circulation. 2020 142:2045-2059). Consequently, highly cellular neointimal lesions arise, occupying luminal space. While IH is complex involving many cell types and events, recent studies applying lineage tracing and single-cell sequencing support that phenotypic changes of resident SMCs play a central role in IH-associated diseases (Alencar G F, et al. Circulation. 2020 142:2045-2059; Shankman L S, et al. Nat Med. 2015 21:628-37; Wu W, et al. Arterioscler Thromb Vasc Biol. 2020 40:1870-1890).

A fraction of transcription factors (TFs), so-called master TFs, are critically important for maintaining or switching cell phenotypes in various sources of cells (Whyte W A, et al. Cell. 2013 153:307-19; Bradner J E, et al. Cell. 2017 168:629-643). In SMCs, KLF4 strongly promotes cell de-differentiation by inhibiting the expression of SMC markers (contractile proteins) (Liu Y, et al. J Biol Chem. 2005 280:9719-27). Another master TF, c-MYB, has been well documented to perpetuate SMC proliferation and migration, and also IH in animals (You X M, et al. Circ Res. 2003 92:314-21; Khachigian L M. Int J Mol Sci. 2019 20; Simons M, et al. Nature. 1992 359:67-70). In this light, blocking IH-driving master TFs would effectively abrogate IH (Khachigian L M. Int J Mol Sci. 2019 20). However, TFs are not typical drug targets, and currently, small-molecule inhibitors are not available for most of TFs including c-MYB and KLF4. This underscores the importance of alternative approaches to targeting TFs.

In the past decade, microRNAs (miRs) have emerged as promising candidate therapeutics (Hanna J, et al. Front Genet. 2019 10:478). This class of small non-coding RNAs is posttranscriptional regulators closely involved in development and disease (Bhaskaran M and Mohan MVet Pathol. 2014 51:759-74; Mitchell P S, et al. Proc Natl Acad Sci USA. 2008 105:10513-8; Rupaimoole R and Slack F J. Nat Rev Drug Discov. 2017 16:203-222; Wilczynska A and Bushell M. Cell Death Differ. 2015 22:22-33; Zampetaki A, et al. J Am Coll Cardiol. 2012 60:290-9). They negatively regulate gene expression through complementary sequences in the 3′UTR of the target gene mRNA. Some miRs were recently found to be key regulators of IH and have attracted considerable attention in vascular research (Zampetaki A, et al. J Am Coll Cardiol. 2012 60:290-9; Cheng Y, et al. Circ Res. 2009 105:158-66; Liu X, et al. Circ Res. 2009 104:476-87; Hergenreider E, et al. Nat Cell Biol. 2012 14:249-56; Liu N, et al. Genes Dev. 2008 22:3242-54; Schulte C, et al. PLoS One. 2015 10:e0145930; Tang Y, et al. Am J Physiol Heart Circ Physiol. 2017 313:H641-H649; Du M, et al. iScience. 2022 25:104169; Farina F M, et al. Circ Res. 2020 126:e120-e135). For example, the miR145 cluster and the miR200 cluster help maintain the SMC contractile phenotype (Cheng Y, et al. Circ Res. 2009 105:158-66; Du M, et al. iScience. 2022 25:104169). On the other hand, miR21 promotes SMC phenotypic switching exacerbating injury-induced IH (Wang D, et al. Arterioscler Thromb Vasc Biol. 2015 35:1945-53). In a translational endeavor using a humanized model, the implantation of stents coated with anti-miR against miR21 reduced IH (Wang D, et al. Arterioscler Thromb Vasc Biol. 2015 35:1945-53).

However, there remains the lack of an approved siRNA or miRNA therapy for cardiovascular disease because of several major hurdles. For example, naked siRNAs or miRNAs delivered in the circulation do not aggregate at the site of the lesion in the vessel wall because they are sequestered by the major organs including the liver. Moreover, siRNAs/miRNAs can be immunogenic and rapidly destroyed by the internal immune system.

### SUMMARY

Disclosed herein is a biomimetic torpedo that can circumvent existing hurdles for RNA therapies. That is, RNA therapeutics can be harbored inside a torpedo shell made of neutrophil membranes in hybrid with a liposome membrane, which enables lesion-targeting and shielding from immunogenicity. Surprisingly, biomimetic torpedoes are only minimally detected or sequestered in major mouse organs including the liver, heart, kidney, and spleen. This is a detriment of other biomimetic approaches—the particles are sequestered in the major organs and do not make their way to the site of arterial injury or arterial inflammatory damage. In preclinical and clinical studies, a general problem is that intravenously delivered therapeutic agents are mostly trapped in major organs, whether they are delivered in liposomes or polymer nanoparticles or biomembrane-camouflaged particles. Moreover, the compatible payloads of the biomimetic torpedo can be any nucleotide agents, whether siRNA, miRNA, aptamers, sgRNA, antisense RNA, long non-coding RNA, plasmid DNA or mRNA.

Therefore, disclosed herein is a biomimetic targeting system that involves a neutrophil cell membrane capsule that encapsulates a therapeutic RNA. In some embodiments, the system further contains a plurality of peptides that specifically target collagen-IV inserted into the neutrophil cell membrane capsule. In some embodiments, the peptide has the amino acid sequence SEQ ID NO:15. In some embodiments, the peptide is conjugated to a cholesterol molecule for integration into the neutrophil cell membrane.

In some embodiments, the therapeutic RNA or DNA is an siRNA, shRNA, mRNA, sgRNA, antisense RNA, long non-coding RNA, miRNA, or plasmid DNA. For example, in some embodiments the therapeutic RNA is miR579-3p and in some embodiments the therapeutic RNA has the nucleic acid sequence UUCAUUUGGUAUAAACCGCGAUU (SEQ ID NO:16).

In some embodiments, the therapeutic RNA is an ALDH1A3-specific siRNA, BRD4-specific siRNA, BRD2-specific siRNA, EZH2-specific siRNA, EED-specific siRNA, DOT1L-specific siRNA, BAHCC1-specific siRNA, HDAC6-specific siRNA, TMEM97-specific siRNA, AURKB-specific siRNA, PLK4-specific siRNA, KLF4-specific siRNA, c-MYC-specific siRNA, or PERK-specific siRNA.

In some embodiments, the cell membrane capsule prepared using neutrophils, macrophages, leukocytes, or mesenchymal stem cells, or exosome capsule extracted from these cells, further contains liposome lipids. For example, in some embodiments, the capsule is produced by a process that involves fusing a liposome encapsulating the therapeutic RNA or DNA with a neutrophil biomembrane.

In some embodiments, the cell membrane capsule encapsulates a polymeric nanoparticle loaded with the RNA or DNA therapeutic.

Also disclosed is a method for preventing restenosis in a subject that involves administering to the subject an effective amount of the targeted gene therapy system disclosed herein.

In some embodiments, the targeted gene therapy system is administered within 1, 2, 3, 4, 5, 6, 7, 8, 9 or 10 days of an angioplasty treatment. In some embodiments, the subject is not given a stent.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1C show treatment of injured rat carotid arteries with miR579-3p-expressing lentivirus inhibits injury-induced IH. FIG. 1A shows in vitro experiments showing down-regulation of miR579-3p in AoSMCs by a panel of pro-IH cytokines. AoSMCs were starved in basal medium (no FBS) for 24 h and then treated for 24 h without (Mock control) or with a cytokine (50 ng/ml PDGF-BB, 20 ng/mL TGFβ1, 20 ng/ml TNFα, or 10 ng/ml IL1β). miR579-3p mRNA was measured by qRT-PCR. Data are presented as mean±SD (n=3 replicates). ^{###}P<0.001 compared to the Mock control, as analyzed with one-way ANOVA followed by Bonferroni post hoc test. FIGS. 1B and 1C show in vivo studies showing inhibition of IH by the treatment with miR579-3p-expressing lentivirus. IH was induced by balloon injury in the rat common carotid artery, and the lentivirus used to express scrambled microRNA or miR579 was locally infused into the injured artery wall. Arteries were collected at post-injury day 14 for histology and morphometric analyses. Representative H&E-stained artery cross-sections are shown in FIG. 1B. A pair of arrows define the neointima thickness. A, adventitia; M, media; N, neointima. Scale bar: 100 μm (Low mag.), 20 μm (High mag). Quantitative morphometric analysis (C): IH=neointima vs media (I/M) area ratio, normalized neointima thickness, lumen area, stenosis rate, and EEL perimeter. The data values of three sections from each animal were averaged. The averages from seven animals in each treatment group were then averaged again to produce mean±SEM. *p<0.05, n=7 rats, unpaired Student's t-test.

FIGS. 2A to 2E show miR579-3p suppresses AoSMC proliferation, migration, and de-differentiation. FIGS. 2A and 2B show proliferation assays. AoSMCs starved in basal medium (no FBS, 24 h) were transfected with miR-con or miR579-3p for 24 h. The medium was then changed to fresh basal medium with or without a cytokine (FIG. 2A) (50 ng/ml PDGF-BB, 20 ng/mL TGFβ1, 20 ng/ml TNFα, or 10 ng/ml IL1β) or full medium (FIG. 2B) for an additional 24 h before the CellTiter-Glo viability assay. FIG. 2C show results of a migration assay. AoSMCs were seeded in the Transwell insert, the lower chamber filled with full medium. Cells that migrated to the lower surface of the insert were imaged after a 24 h incubation. To quantify the migration, 33% (v/v) Acetic acid was added into the insert to elute the bound crystal violet, and then the eluent from the lower chamber was measured for absorbance (590 nm) using a 96-well microplate reader. FIG. 2D show de-differentiation (Western blotting of SMC contractile proteins). AoSMCs were starved (no FBS, 24 h) and transfected with miR-con or miR579-3P for 24 h, and then treated with or without 50 ng/ml PDGF-BB for another 24 h. FIG. 2E show immunofluorescence of SMC contractile proteins. AoSMCs were cultured and treated with PDGF-BB as described in FIG. 2D. Cells were fixed with 4% paraformaldehyde followed by permeabilization and staining with primary antibodies. Fluorophore-conjugated secondary antibodies were applied followed by counterstaining with DAPI and confocal imaging. Quantification (FIGS. 2A-2D): Mean±SD (A and B, see dot plots for the number of replicates) or mean±SEM (FIGS. 2C and 2D, n=3 repeats). Pairwise comparison was made through Student's t-test. **P<0.01, ***P<0.001.

FIGS. 3A to 3L show miR579-3p represses the expression of c-MYB and proliferation/migration marker proteins. AoSMCs starved in basal medium (no FBS, 24 h) were transfected with miR-con or miR579-3p for 24 h. The medium was then changed to fresh basal medium with or without a cytokine (50 ng/ml PDGF-BB, 20 ng/mL TGFβ1, 20 ng/ml TNFα, or 10 ng/ml IL1β) or full medium (migration assay) for an additional 24 h prior to various assays. FIG. 3A is a legend of symbols for FIGS. 3B-31. FIG. 3B shows qRT-PCR showing upregulation of c-MYB mRNA by cytokines. FIG. 3C shows qRT-PCR showing negative regulation of c-MYB mRNA by miR579-3p. FIGS. 3D-31 show Western blots showing that miR579-3P negatively regulates protein levels of c-MYB and proliferation/migration markers. FIG. 3J shows Western blots showing that c-MYB overexpression increases proliferation/migration markers. FIG. 3K shows proliferation assay indicating that c-MYB overexpression rescues miR579-3P-mitigated AoSMC proliferation. PDGF-BB was included in the SMC culture. FIG. 3L shows transwell assay indicating that c-MYB overexpression rescues miR579-3P-inhibited AoSMC migration. Quantification (FIGS. 3A-3L): Data are presented as mean±SD (qRT-PCR, n=3 replicates) or mean±SEM (Western blots, proliferation, and migration, n=3 repeats); ^{###}P<0.001 (compared to Mock control, the first bar), as analyzed with one-way ANOVA followed by Bonferroni post hoc test. Pairwise comparison was made through Student's t-test: *P<0.05, **P<0.01, ***P<0.001.

FIGS. 4A to 4C show miR579-3p directly targets the c-MYB mRNA 3′UTR. The cDNA of wild type 3′-UTR of c-MYB was cloned from the human genome. The sequences of wild type and mutants with 12 bp deletion at position 1, 11 bp deletion at position 2, or deletion of both were amplified and subcloned into the XhoI and NotI sites between Renilla gene and polyA of the psiCheck2 vector. Each constructed luciferase reporter plasmid or empty vector was transfected into AoSMCs (5000 cells per well) in 96-well plates. Luciferase was assayed at 48 hours post transfection using the Dual-Luciferase Reporter Assay System. Luminescence was quantitated and renilla luciferase readings were normalized against the firefly luciferase activity to determine the relative luciferase activity. Data are presented as mean±SD (n=3 replicates). Pairwise comparison was made through Student's t-test: ***P<0.001; n.s., not significant. FIG. 4A is a schematic of the luciferase reporter vector and constructs. FIG. 4B shows the miR579-3p sequences complementary to that at positions 1 and 2 of c-MYB 3′UTR. FIG. 4C shows relative luciferase activity compared between miR579-3p and miR-Con.

FIGS. 5A to 5C show miR579-3p targets the KLF4 mRNA 3′UTR. Wild type and 13 bp deletion of 3′-UTR of KLF4, was amplified from the human genome and subcloned into the XhoI and NotI sites between Renilla gene and polyA of the psiCheck2 vector. Each constructed luciferase reporter plasmid or empty vector was transfected into AoSMCs at 5000 cells per well in 96-well plates. Luciferase was assayed at post 48 hours transfection using the Dual-Luciferase Reporter Assay System. Luminescence was quantitated and renilla luciferase readings were normalized against the firefly luciferase activity to determine the relative luciferase activity. Data are presented as mean±SD (n=3 replicates). Pairwise comparison was made through Student's t-test: ***P<0.001; n.s., not significant. FIG. 5A is a schematic of the luciferase reporter vector and constructs. FIG. 5B shows the miR579-3p sequence complementary to that of the KLF4 3′UTR. FIG. 5C shows relative luciferase activity compared between miR579-3p and miR-Con.

FIGS. 6A to 6C show miR579-3p lentiviral treatment of injured rat carotid arteries reduces c-MYB and KLF4 expression and increases SMC contractile proteins. The lentivirus for expressing miR579-3p or miR-Con was infused into the rat carotid artery wall immediately after balloon injury. The arteries were collected 14 days after injury and immunohistochemistry was performed on cross-sections to detect c-MYB, KLF4, αSMA, SM22, Calponin, and MYH11. FIGS. 6A and 6B show representative immunostained cross-sections (low-mag and high-mag, respectively). Negative staining: no primary antibody. Scale bar: 100 μm for low-mag pictures and 20 μm for high-mag pictures. FIG. 6C shows quantification. Colorimetric intensity per image field from 7 animals (3-4 cross-sections per rat) was averaged to calculate mean±SEM (n=7 rats). Statistics: unpaired Student t-test; *P<0.05, **P<0.01, ***P<0.001.

FIGS. 7A to 7C show miR579 and c-MYB gene are inversely correlated under cytokines stimulation. Microarray was performed as described in our previous paper 35. AoSMCs were starved in basal medium (no FBS) for 24 h and then treated for 24 h without (Mock control) or with a cytokine (50 ng/ml PDGF-BB, 20 ng/mL TGFβ1, 20 ng/ml TNFα, or 10 ng/ml IL1β) prior to the microarray experiment. FIGS. 7A and 7B show mir579 and c-MYB expression from microarray analysis without (Mock control) or with stimulation by a cytokine (PDGF-BB, TGFβ, IL1β, or TNFα). FIG. 7C shows fold change of the expression (cytokine stimulated versus Mock) of miR579 and c-MYB. The data from A and B was replotted to show an inversed relation of the changes in miR579 and c-MYB expression stimulated by each cytokine. Mean±SD (n=3 replicates).

FIG. 8 is a schematic of a biomimetic torpedo disclosed herein. A. Biomembranes (isolated from neutrophils or exosomes). B. siRNA (red) loaded in liposome or polymer-based nanoparticle (herein denoted as siRNA carriers). C. Biomembrane fused with liposome (chimeric) or coated on nanoparticle endows these siRNA carriers with a targeting ability (so called Biomimetic Torpedo). D. Biomimetic Torpedo formulations can be tail-vein injected to home in on injured artery sites.

FIGS. 9A to 9D show a schematic design: Biomimetic Torpedo targeting injured artery. FIGS. 9A and 9B show how angioplasty injures the endothelium exposing SMCs, which turn dysfunctional forming IH. FIG. 9C shows a biovelcro design to enhance Biomimetic Torpedo's targeting specificity for injured artery wall. Injury (EC damage) exposes the underlying basement membrane (BM) rich in collagen-IV, i.e. “loops”; whereby Biomimetic Torpedo decorated with Col-IV-binding peptides (“hooks”) can attach to injured sites to deliver siRNA into dysfunctional SMCs and ECs. Thus, injured (BM-exposed) but not normal (EC-covered) arteries provide homing sites for Torpedo. The peptide is conjugated to a flexible PEG arm with cholesterol at the end that naturally inserts into the biomembrane. A=adventitia; M=media; I=intima.

FIGS. 10A to 10D show prototype Biomimetic Torpedo targets injured arteries with no severe entrapment in major organs. FIGS. 10A and 10B show Cy5-siRNA encapsulated in neutrophil membranes (decorated with Col-IV-binding peptide) was tail-vein injected into mice (#2, 3, 4) after wire injury of femoral artery (the contralateral side uninjured). PBS (no Torpedo) was injected into mouse #1. FIGS. 10C and 10D show Torpedo accumulation on injured but not uninjured arteries (*), with only basal-level in main organs. IVIS Cy5 detection (at 24 h post injection) was normalized as per cm² surface of radiance (sr) in FIG. 10B, and normalized to organ weight in FIG. 10D. CFA: common femoral artery. Kid: kidney. L-CFA is injured artery, R-CFA is uninjured artery. Quantification: Mean±SEM, n=5 mice. ANOVA/Tukey: ***P<0.001, each compared to injured artery (i.e. the first value bar).

FIG. 11 shows ALDH1A3 silencing mitigates IH. Following angioplasty, lentivirus was infused locally into the injured rat carotid artery wall to express scrambled (Scr) or ALDH1A3 shRNA. A, adventitia; M, media; I, intima (between arrows). IH (I/M area ratio) was measured on artery sections of post-injury day-14. Mean±SEM, n=4-5 rats (see data points). Student t-test: *p<0.05. Scale: 100 μm. Immunostaining: ALDH1A3 decreased in artery wall after sh/siRNA silencing. si-1A3 is ALDH1A3-specific siRNA.

FIGS. 12A and 12B show ALDH1A3 silencing promotes re-endothelialization. The endothelium was damaged by balloon angioplasty in rat common carotid arteries (Evans Blue-stained, as far as arrow-pointed). Lentivirus was infused locally into the damaged artery wall to express ALDH1A3 shRNA (siALDH1A3 as final product). Treated arteries recovered faster (unstained area expanding above the line) than scrambled (Scr) controls (at the line). Mean±SEM, n=7 rats (data points in FIG. 12A). Unpaired student t-test: *p<0.05. si-1A3 is ALDH1A3-specific siRNA.

FIG. 13 shows ALDH1A3 silencing in ECs reduces pro-inflammatory cytokine expression. Human aortic ECs were siRNA-transfected and TNFα-treated Mean±SEM, n=3 repeat exp. ANOVA/Tukey: *P<0.05.

FIG. 14 shows knockdown of 1A3 mitigates IH. Following angioplasty, lentivirus was infused locally into the injured rat carotid artery wall to express scrambled (Scr) or 1A3 shRNA. A, adventitia; M, media; N, neointima (between arrows). IH is N/M area ratio (post-injury day-14). Mean±SEM, n=4-6 rats. Student t-test: *p<0.05. Scale: 100 μm. Immunostain-ing confirms 1A3 knockdown.

FIGS. 15A and 15B show knockdown of 1A3 promotes EC recovery at scratch wound. Human aortic ECs transfected with scrambled or 1A3-specific siRNA were treated with or without TNFα (20 ng/ml). FIG. 15A contains images at 24 h after scratch. FIG. 15B shows gap width. Mean±SEM, n=3 repeat experiments, One-way ANOVA/Tukey test: **P<0.01. Note: Wound (gap) healing requires both EC growth (proliferation) and migration.

FIG. 16 shows knockdown of 1A3 up-regulates EC-protective genes and down-regulates EC-detrimental genes. Human aortic ECs transfected with scrambled or 1A3-specific siRNA were treated with TNFα (20 ng/ml, 24 h), triplicate samples (n=3) were used for RNA sequencing.

FIGS. 17A and 17B show knockdown of 1A3 in ECs increases EC-protective factors (FIG. 17A) and reduces the expression of EC-detrimental factors (FIG. 17B). Human aortic ECs were transfected with 1A3-specific siRNA (si-1A3) or scrambled (Scr) control, and then treated without or with TNFα (20 ng/ml, 24 h). Mean±SEM, n=3 independent experiments. ANOVA/Tukey test: *P<0.05.

FIG. 18 shows immunostained 1A3 in the EC nucleus and cytosol. Negative staining: No primary antibody. Scale bar: 10 μm.

FIG. 19 shows increase of 1A3 in the neointima of human diseased (vs normal) coronary arteries. Sections were from CVPath Institute Inc, MD. A, adventitia; M, media; N, neointima (between arrows). Mean±SEM, n=5 patients. Stu-dent's t-test:*p<0.05. Stained 1A3; Nuclei.

FIGS. 20A to 20D shows a schematic design: Biomimetic Torpedo targeting injured arteries. FIGS. 20A and 20B show angioplasty injures the endothelium exposing the under-lying matrix layer rich in collagen-IV (Col4), where Biomimetic Torpedo decorated with Col4-binding peptides can attach to and deliver siRNAs into dysfunctional ECs and SMCs. The peptide is linked with cholesterol which, as a common component of biomembranes, naturally inserts into the membrane. FIG. 20C contains the legends.

FIG. 20D shows an electromicroscopy (EM) image of Biomimetic Torpedo.

FIGS. 21A to 21C shows Biomimetic Torpedo targets injured mouse femoral arteries with minimal entrapment in major organs. Torpedo: Cy5-siRNA encapsulated in the hybridized membrane (neutro-phil+liposome) inserted with Col4-binding peptides. It was tail-vein injected after wire injury. FIG. 21A is an IVIS image (24 h after injection). *Arteries. FIG. 21B shows an enlarged view. FIG. 21C shows fluorescence normalized to organ weight. Mean±SEM, n=5 mice. ANOVA/Tukey test: ***P<0.001, each vs the red bar.

FIGS. 22A and 22B show Biomimetic Torpedo accumulates in balloon angioplasty-wounded (but not intact rat common carotid arteries). The right-side artery was partially injured (dashed line); the left side was not. FIG. 22A shows rats tail-vein injected with exosome-membrane Torpedo containing Cy5-labeled 1A3 siRNA and IVIS (In Vivo Imaging Systems, red signal) was applied 24 h after injection. FIG. 22B shows a control. Rats were injected with free naked Cy5-labeled 1A3 siRNA only (no Torpedo).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### SPECIFIC EMBODIMENTS

Embodiment 1. A biomimetic targeting system, comprising a neutrophil cell membrane capsule that encapsulates a therapeutic RNA.

Embodiment 2. The system of embodiment 1, further comprising a plurality of peptides that specifically target collagen-IV inserted into the neutrophil cell membrane capsule.

Embodiment 3. The system of embodiment 2, wherein the peptide comprises the amino acid sequence SEQ ID NO:15.

Embodiment 4. The system of embodiment 2 or 3, wherein the peptide is conjugated to a cholesterol molecule for integration into the neutrophil cell membrane.

Embodiment 5. The system of any one of embodiments 1 to 4, wherein the therapeutic RNA is an siRNA, shRNA, mRNA, or miRNA.

Embodiment 6. The system of embodiment 5, wherein the therapeutic RNA comprises miR579-3p.

Embodiment 7. The system of embodiment 6, wherein the therapeutic RNA comprises the nucleic acid sequence UUCAUUUGGUAUAAACCGCGAUU (SEQ ID NO:16).

Embodiment 8. The system of embodiment 5, wherein the therapeutic RNA is an ALDH1A3-specific siRNA.

Embodiment 9. The system of any one of embodiments 1 to 8, wherein the neutrophil cell membrane capsule further comprises liposome lipids.

Embodiment 10. The system of embodiment 9, wherein the capsule is produced by a process comprising fusing a liposome encapsulating the therapeutic RNA with a neutrophil biomembrane.

Embodiment 11. The system of any one of embodiments 1 to 8, wherein the neutrophil cell membrane capsule encapsulates a polymeric nanoparticle loaded with the RNA therapeutic.

Embodiment 12. A method for preventing restenosis in a subject, comprising administering to the subject an effective amount of the targeted gene therapy system of any one of embodiments 1 to 11.

Embodiment 13. The method of embodiment 12, wherein the targeted gene therapy system is administered within 1 day of an angioplasty treatment.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: MiR579-3p is a Novel Neointima Inhibitory Modulator that Directly Targets Master Transcription Factors c-MYB and KLF4

In a previous study (Xie X, et al. Cell Death Discov. 2021 7:318), through unbiased analysis of microarray and bioinformatics, an approach was established to screen miRs that are responsive to each of the 4 salient cytokines that surge after vascular injury, namely, PDGF-BB, TGFβ1, TNFα, and IL1β. On this basis, miR579-3p was identified as a novel negative regulator of SMC phenotypic switching. Interestingly, these data demonstrated that miR579-3p targets not only c-MYB but also KLF4. Importantly, in a therapeutic evaluation using a rat model of injury-induced IH, treating injured arteries with lentivirus to express miR579-3p significantly reduced IH. These findings together demonstrate that miR579-3p is a novel modulator for preserving SMC phenotypic stability, suggesting a new option for translational development to combat IH and associated diseases.

### Materials and Methods

### Materials

Human Aortic smooth muscle cells (AoSMCs, CC-2571), smooth muscle cell basal medium (SmBM, CC-3181), and SmBM plus SingleQuots of supplements (CC-3182) were purchased from Lonza. Dulbecco's modified Eagle's medium (DMEM, 11965118) was from Invitrogen. Lenti-X™ 293T cell line was from Clontech (632180). Recombinant Human TGF-beta 1 (240-B), TNF-alpha (210-TA), IL-1 beta (201-LB), and PDGF-BB (520-BB) were from R&D Systems. Cell Titer-Glo 2.0 Assay kit (G9242), psiCHECK2 vector (C8021), and Dual-Luciferase Reporter Assay System (E1910) were from Promega. For ImmPRESS™ HRP Anti-Rabbit IgG (Peroxidase) Polymer Detection Kit (MP-7451-15) and ImmPACT DAB Peroxidase (HRP), Substrate (SK-4105) were purchased from Vector Laboratories. Transwell (12-mm diameter, 3.0 μm pore size) Polycarbonate Membrane Insert was from Corning (3402). The following products were from Thermo Fisher Scientific: Scrambled microRNA control (AM4635), hsa-miR-579-3p (Assay ID: MC12340), Opti-MEM I Reduced Serum Medium (31985062), Lipofectamine RNAiMAX Transfection Reagent (13778150), TaqMan MicroRNA reverse transcription kit (4366596), TaqMan Universal Master Mix II (4440043), TaqMan primers (hsa-miR579, Assay ID: 002398; RNU44, Assay ID: 001094), High-Capacity cDNA Reverse Transcription kit (4368814), TaqMan MicroRNA reverse transcription kit (4366596), PowerUp SYBR Green Master Mix (A25778), and TaqMan Universal Master Mix II (4440043). pLenti-Ill-mir Control Vector (m003) and pLenti-mir579-3p (mh10891) were from Applied Biological Materials Inc. c-MYB Lentiviral Particles were from Santa Cruz (sc-400752-LAC). QuikChange II XL Site-Directed Mutagenesis Kit was purchased from Agilent (200521).

### Vascular Smooth Muscle Cell Culture

AoSMCs were cultured in SmBM with supplements. Cells were passaged every 3-4 days at a ratio of 1:4. Cells used in this study were below passage 8. Cultured cells were maintained in a humidified incubator with 5% CO₂ at 37° C.

Quantitative Real-Time PCR (qRT-PCR)

Total RNA was extracted from cell lysates using the TRIzol reagent and then reversed to cDNA using the High-Capacity cDNA Reverse Transcription kit (4368814) or the TaqMan MicroRNA reverse transcription kit (4366596). In each 20 μl reaction, 10 ng of cDNA was amplified through quantitative real-time PCR using PowerUp SYBR Green Master Mix (A25778) or TaqMan Universal Master Mix II (4440043). The mRNA expression of c-MYB and miR579 were determined using the Applied Biosystems 7900HT Fast Real-Time PCR System with validated qPCR or Taqman primers (ThermoFisher Scientific). The mRNA level of c-MYB was normalized to glyceraldehyde 3-phosphate dehydrogenase (GAPDH) and that of miR579 was normalized to RNU44 using the ΔΔCt method. qRT-PCR primers are listed in Table 1. qRT-PCR was performed in triplicate reactions.

### Western Blotting

Cells were lysed in RIPA buffer (50 mM Tris, 150 mM NaCl, 1% Nonidet P-40 and 0.1% sodium dodecyl sulfate) containing Halt™ Protease and Phosphatase Inhibitor Cocktail (Thermo Fisher Scientific, 78440). Protein concentration was determined using a Pierce BCA Protein Assay kit (Thermo Fisher Scientific, 23227). Whole-cell lysates were mixed with Laemmli loading buffer, boiled, separated by 12% SDS-PAGE, and transferred to a PVDF membrane. Subsequently, immunoblot analyses were performed using specific antibodies (source companies, catalog numbers, and dilution ratios listed in Table 2). The signal was developed using the Western Blotting Kit (Pierce, 35050).

### Luciferase Reporter Construction and Assay

The 1.2 kb 3′-UTR of c-MYB was amplified from the human genome and subcloned into the XhoI and NotI sites of the psiCheck2 vector (Promega, C8021). Sigle deletion at position 1 (629-643) or position 2 (873-885) and double deletions at both positions 1 and 2 were generated using QuikChange II XL Site-Directed Mutagenesis Kit (Agilent, 200521). For KLF4 3′UTR construction, the 900 bp 3′-UTR of KLF4 was amplified and subcloned into the XhoI and NotI sites of the psiCheck2 vector. The deletion at 692-704 was generated using QuikChange II XL Site-Directed Mutagenesis Kit. The primers for cloning are presented in Table 3. Correct sequences of the clones were verified through sequencing. We then transfected the cells with the luciferase assay plasmids (The set of c-MYB 3′UTR: empty vector, wildtype 3′UTR, single deletion 1, single deletion 2, or double deletions plasmid; the set of KLF4: empty vector, wildtype 3′UTR, deletion plasmid) using lipofectamine 3000 following the manufacturer's instruction. Briefly, 5000 AoSMCs were seeded in each well of 96-well plates and cultured for 24 h in full medium, then the medium was changed to basal medium 2 hours before transfection. Luciferase assay was performed 48 hours after transfection by following the manufacturer's instruction and using Dual-Luciferase Reporter Assay System (Promega, E1910). Luminescence was quantitated and renilla luciferase readings were normalized against the firefly luciferase activity to determine the relative luciferase activity.

miR579-3p and Cytokines Treatment

For the transfection with miRs, AoSMCs were cultured in the full medium until 90% confluence and changed to basal medium (0% fetal bovine serum) 2 hours before transfection. The cells were then transfected with hsa-miR579-3p or scrambled control using Lipofectamine RNAiMAX Transfection Reagent (following the manufacturer's instructions) for 12 hours and cultured with fresh basal medium (no Lipofectamine) for another 12 hours. PDGF-BB (50 ng/ml), TGFβ1 (20 ng/mL), TNFα (20 ng/ml), or IL1β (10 ng/ml) was then added, and cells were harvested for assays 24 hours after the cytokine treatment.

c-MYB Over-Expression in AoSMCs

AoSMCs were cultured in the full medium until 70% confluence and changed to basal medium (0% fetal bovine serum) 2 hours before infection. The cells were infected with c-MYB Lentiviral Particles (sc-400752-LAC) at MOI 10 for 12 hours. The medium was changed to either full medium or basal medium depending on the experimental design. After 48 hours of infection, cells in the full medium were collected for Western blot assay. Cells in the basal medium were transfected with miR579-3p for 12 hours before cytokine treatments for additional 24 hours.

### Cell Titer-Glo Viability Assay

AoSMCs of equal number were seeded in each well of the 96-well plate. Cells were treated under conditions specified in figure legends. Cells were washed with PBS once, and then 50 μl of PBS and 50 μl of CellTiter-Glo reagent were added to each well. The 96-well plate was then analyzed for cell proliferation using BioTek Gene 5 Microplate Reader (BioTek Instruments, Inc).

### Transwell Migration Assay

AoSMCs were grown in 12-well transwell upper inserts at 80-90% confluence. After 24 hours of starvation, cells were transfected with hsa-miR579-3p or scrambled control miR, or infected with c-MYB Lentiviral Particles. The full medium was added to the bottom of the lower chamber of the transwell. Transwell inserts were washed three times with PBS after 24 hours of migration. The cells on the inside of the Transwell were removed using cotton swabs. The cells on the lower surface of the membrane were stained with 0.1% crystal violet (10% methanol) for 20 min. Washed three times with PBS to remove unbound crystal violet and then air-dried. The migrated cells were imaged under a microscope. Once images were taken, 400 μl 33% (v/v) acetic acid was added to the insert and shaken for 10 min to elute the bound crystal violet. The eluent from the lower chamber was measured for absorbance (590 nm) using a 96-well microplate reader.

### Animals

Male Sprague-Dawley rats were purchased from Charles River Laboratories (Wilmington, MA). All animal experiments were carried out by the recommendations in the Guide for the Care and Use of Laboratory Animals of the National Institutes of Health. The protocol was approved by the Institutional Animal Care and Use Committee (IACUC) of The University of Virginia. Animals were euthanized in a chamber gradually filled with CO₂.

### Rat Carotid Artery Balloon Injury Model

Rat carotid artery balloon injury was performed as we previously described (Wang B, et al. EBioMedicine. 2015 2:1650-1661) with minor modifications. Briefly, after induction of general anesthesia with isoflurane, a 2-French balloon catheter (Edwards Lifesciences Corp., Irvine, CA) was inserted from the left external carotid artery into the common carotid artery of male Sprague-Dawley rats (300-350 g), inflated at a pressure of 1.5 atm, and retracted to the distal bifurcation. This action was repeated three times, and the catheter was rotated while being retracted at the 4^{th} time. Immediately following the surgery, scrambled or miR579-3p lentivirus were filled in the isolated common carotid artery for 25 min. The external carotid artery was ligated, and the blood flow was resumed. The neck incision was then suture-closed and sanitized, and the animal was left on the warm pad to recover. Isoflurane general anesthesia was applied during the surgery (through inhaling, flow rate 2 L/minute). Bupivacaine (up to 8 mg/kg) was locally injected at the incision site, and carprofen (5 mg/kg) and buprenorphine (0.05 mg/kg) were subcutaneously injected after the surgery. The rats were euthanized 14 days after surgery and common carotid arteries were collected after perfusion fixation with 4% paraformaldehyde (PFA). The fixed arteries were processed for sectioning and morphometric analysis.

### Morphometric Analysis of Neointimal Hyperplasia (IH)

Paraffin-embedded arteries were cut into 5-μm sections for hematoxylin-eosin (H&E) staining. Lumen area, area inside internal elastic lamina (IEL), and the area inside the outer boundary of adventitia together with lumen and IEL perimeters were measured on the sections and calculated using ImageJ. Calculations were conducted following previous publication (Shirasu T, et al. Biomaterials. 2021 275:120968); neointimal area=IEL area−lumen area; normalized intimal thickness=neointima area/IEL perimeter; stenosis rate=(neointima area/IEL area)*100; neointima/media ratio (I/M ratio)=neointima area/(area inside adventitia−IEL area). The data was generated by averaging 2-3 artery sections from each animal, and then the means from seven animals in each treatment group were averaged to get mean±SEM.

### Immunofluorescence

Transfected cells were fixed with 4% paraformaldehyde followed by permeabilization and staining with primary antibodies for KLF4, SMA, SM22, Calponin, or MYH11. The secondary antibodies used were Goat Anti-Rabbit IgG Antibody DyLight 488 (Vector Laboratories, DI-1488) and DyLight 594 (Vector Laboratories, DI-1094), Cell nuclei were counterstained with an antifade mounting medium containing DAPI (Vector Laboratories, H-1200). Cells were imaged using an inverted confocal microscope (Leica LSM8100 Confocal Microscope, Leica Microsystems)

### Immunohistochemistry

IHC was performed as previously described (Xie X, et al. iScience. 2019 19:872-882). In brief, paraffin-embedded arteries were cut into 5-μm sections for immunostaining analysis. Artery slides were deparaffinized and rehydrated through xylenes and graded alcohol series. Antigen retrieval was done using citrate buffer for 2 hours at 80° C. in a high-pressure cocker. Endogenous peroxidase was blocked by incubation with 3% H₂O₂ for 15 min. ImmPRESS™ HRP Anti-Rabbit IgG (Peroxidase) Polymer Detection Kit (Vector Laboratories, MP-7451-15) was used to perform immunostaining of c-MYB, KLF4, αSMA, SM22, Calponin, and MYH11. The staining was visualized using ImmPACT DAB Peroxidase (HRP) Substrate. Six different fields were then imaged from each section.

### Statistical Analysis

Values were expressed as mean±SEM derived from independent experiments or mean±SD of replicates. Statistical analyses were performed with Prism 8.0 (GraphPad Software). Comparison between experimental groups was analyzed by Student's t-test or one-way ANOVA, as specified in figure legends. P values<0.05 were considered statistically significant.

### Results

miR579 is a Novel IH-Mitigating Modulator

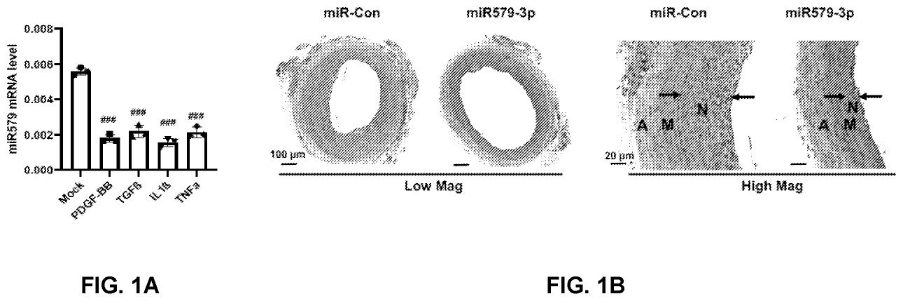

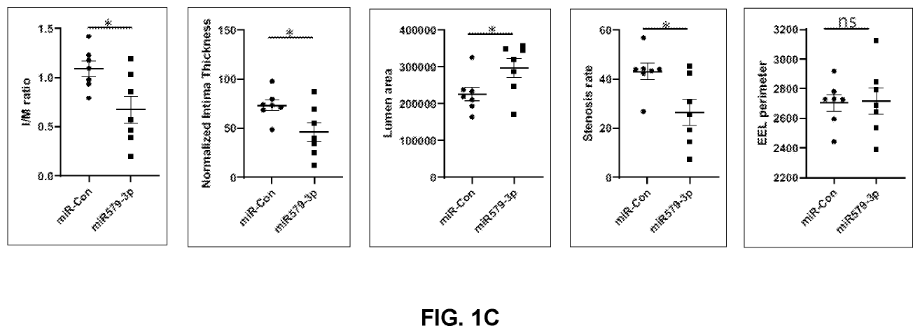

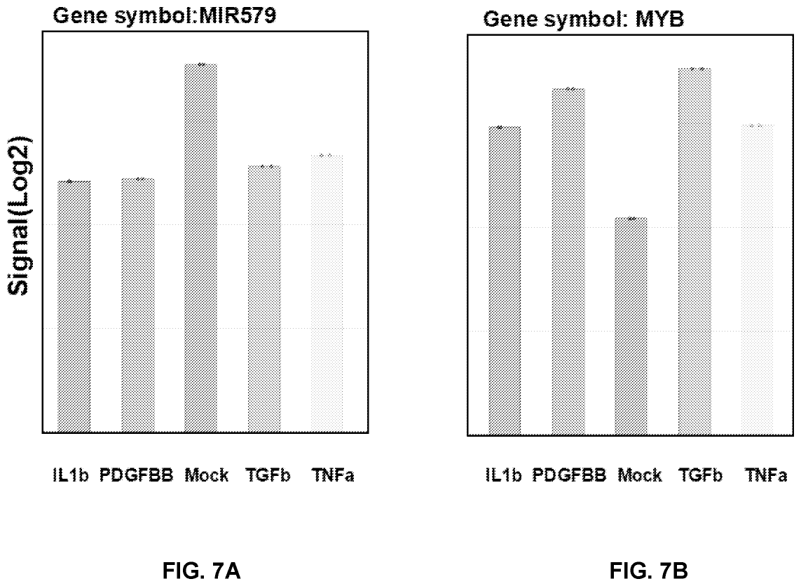

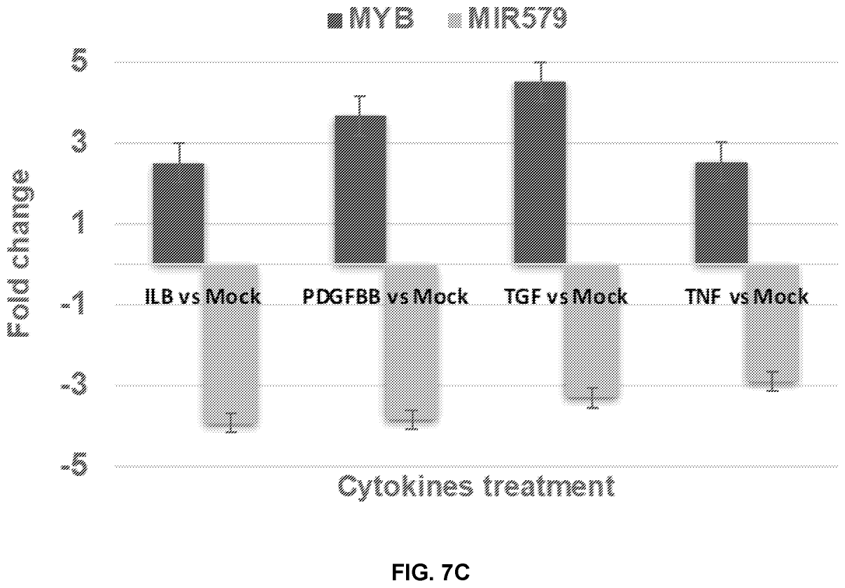

Common vascular reconstructive procedures, whether angioplasty or bypass grafting, expose the medial SMCs to a myriad of cytokine stimuli that trigger IH-forming SMC phenotypic changes, salient cytokines including PDGF-BB, TGFβ1, TNFα, and IL1β. PDGF-BB has been widely used to stimulate SMC phenotypic switching in cell culture. However, the microenvironment of SMCs in the damaged vascular wall is complex, and so is IH pathogenesis. Therefore, we adopted a more holistic approach in a previous microarray study (Xie X, et al. Cell Death Discov. 2021 7:318) to screen miRNAs that robustly respond to all 4 cytokines (see above) rather than PDGF-BB alone. Via unbiased screening and bioinformatics, herein we identified a novel regulator of SMC plasticity, miR579-3p, which was down-regulated by either of the 4 cytokines (FIGS. 1A, 7A). There are only a very small number of reports related to miR579-3p, mainly in cancer cells (Xu J, et al. Aging (Albany NY). 2021 13:16471-16484; Barbier R H, et al. Sci Rep. 2021 11:10765; He X, et al. Am J Transl Res. 2021 13:8777-8786; Fattore L, et al. Proc Natl Acad Sci USA. 2016 113:E5005-13). Its function in SMCs and IH was not known.

To explore its potential role as an IH regulator, angioplasty was conducted to injure rat carotid arteries thereby inducing IH (Xie X, et al. iScience. 2019 19:872-882), and infused lentivirus locally into the damaged artery wall to express miR579-3p or miR-Con (control microRNA). Morphometric parameters were measured on cross-sections of the arteries collected 14 days after angioplasty (FIG. 1B). As indicated by the quantitative data in FIG. 1C, the treatment with the miR579-3p-expressing lentivirus (vs miR-Con) significantly reduced IH, whether presented as the neointima/media area ratio (I/M) or normalized neointima thickness. Accordingly, stenosis was mitigated, as indicated by significantly increased lumen area and decreased stenosis rate. There was no vessel shrinkage, an adverse outcome, as no change was observed in the overall vessel size (EEL perimeter). Thus, these results uncovered miR579-3p as a novel negative regulator of IH.

miR579-3p is a Novel Negative Regulator of SMC Phenotypic Switching

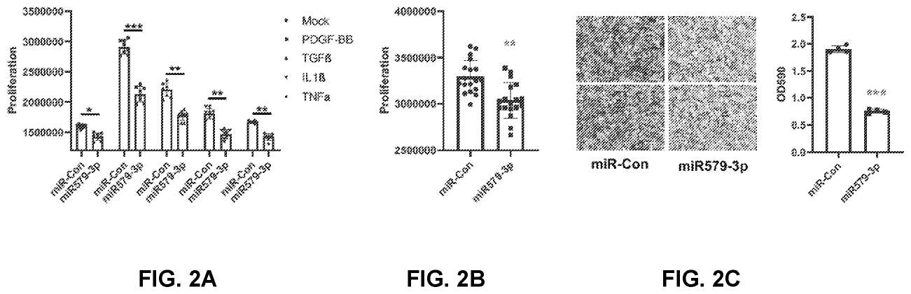

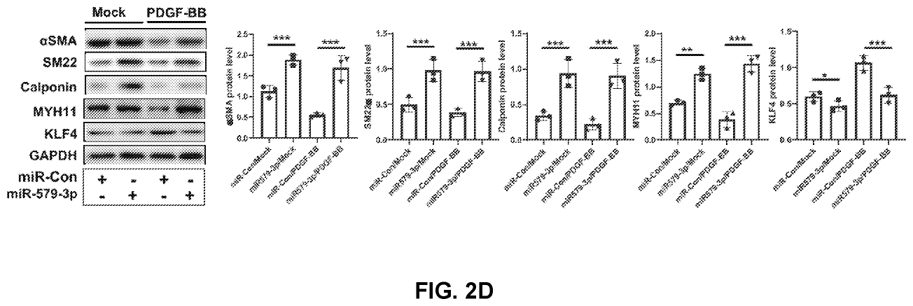

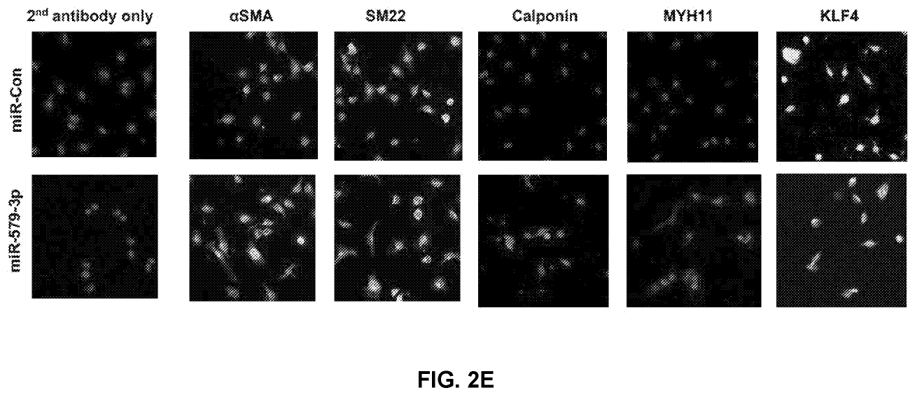

It has been well documented that injury-induced IH is primarily formed by SMCs that have undergone phenotypic switching (Wu W, et al. Arterioscler Thromb Vasc Biol. 2020 40:1870-1890; Alexander M R and Owens G K. Annual review of physiology. 2012 74:13-40). Therefore, next determined was the role of miR579-3p in this process. Increasing miR579-3p in human primary aortic SMCs (AoSMCs) via transfection markedly reduced (vs miR-Con) AoSMC proliferation in the presence of either of the 4 cytokines, namely, PDGF-BB, TGFβ1, TNFα, and IL1β (FIG. 2A). This proliferation-blunting effect was also observed with AoSMCs cultured in full medium (FIG. 2B). Moreover, miR579-3p reduced the migration of AoSMCs cultured in full media by ˜60% (FIG. 2C).

Next determined was the effect of miR579-3p transfection on SMC de-differentiation through Western blotting of a panel of contractile protein markers. Since PDGF-BB among the 4 cytokines is most commonly used to robustly induce SMC de-differentiation, PDGF-BB we chosen as the stimulant for this experiment. Interestingly, the data in FIG. 2D indicates that miR579-3p drastically increased SMC contractile proteins αSMA, SM22, calponin, and MYH11, which were reduced by PDGF-BB in the presence of miR-Con. The effect of miR579-3p on elevating SMC marker proteins was also observed in the absence of PDGF-BB albeit to a lesser extent. Of note, while PDGF-BB upregulated KLF4, the master TF that represses the expression of SMC contractile genes (Yoshida T, et al. Circ Res. 2008 102:1548-57), the miR579-3p transfection of AoSMCs brought KLF4 protein back to the basal level. Further supporting these results, immunofluorescence illustrated that miR579-3p increased SMC contractile proteins and decreased KLF4 (FIG. 2E).

Thus, these results collectively demonstrate that miR579-3p promotes AoSMC proliferation, migration and de-differentiation, we have therefore identified a novel miR regulator of SMC phenotypic switching.

miR579-3p Negatively Regulates c-MYB Expression

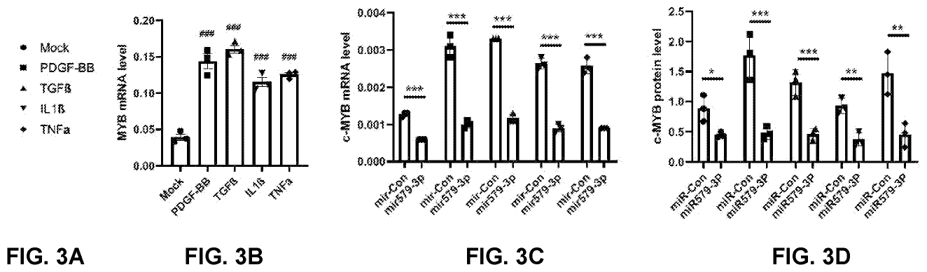

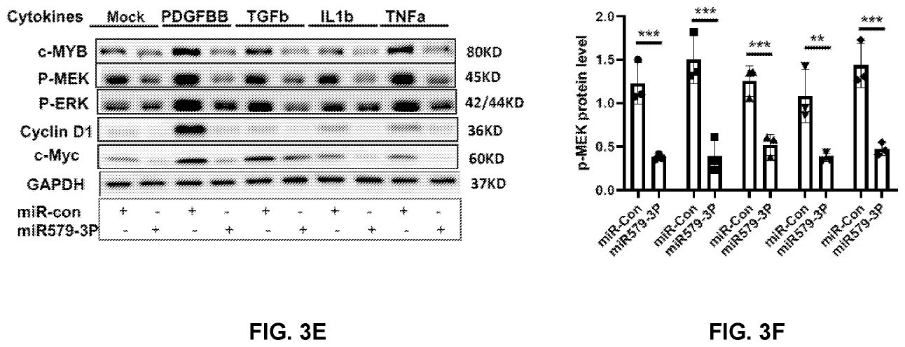

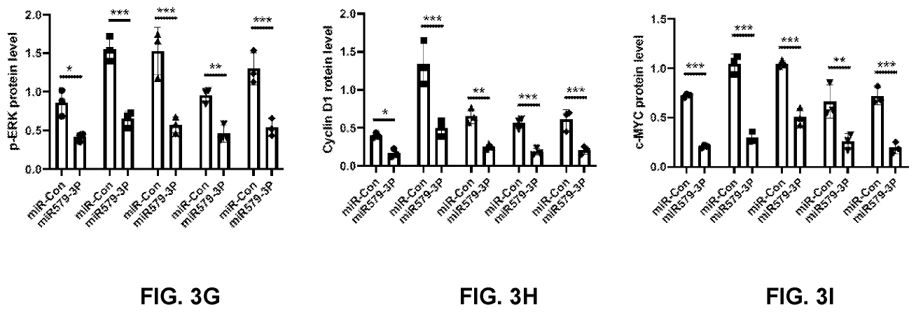

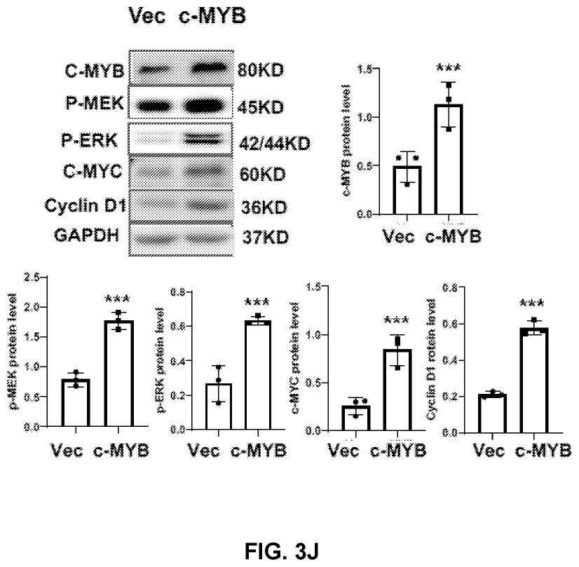

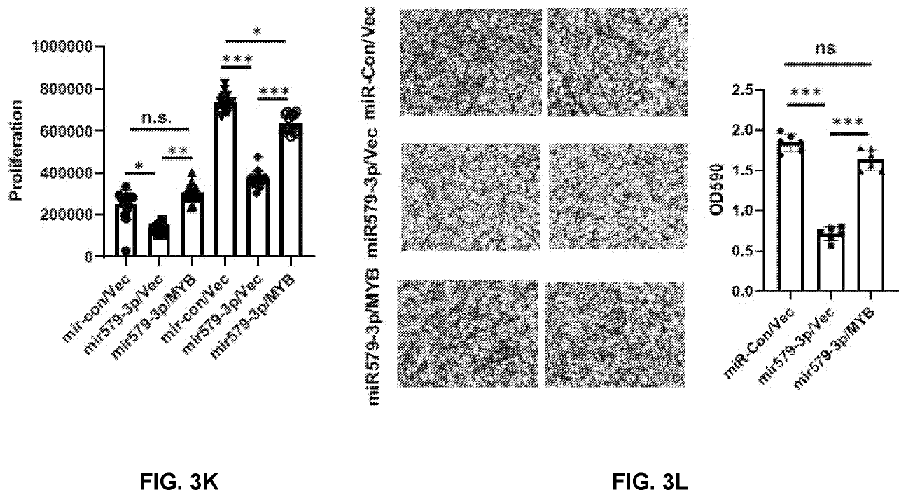

In the pursuit of the molecular mechanisms that underlie the miR579-3p regulation of SMC phenotype, opposite trends of cytokine-induced gene expression of miR579-3p and c-MYB was noticed (FIG. 7B). Whereas miR579-3p was downregulated, c-MYB was upregulated by all 4 cytokines in AoSMCs FIG. 7C). This inversed correlation was verified by qRT-PCR (FIGS. 1A, 3B). While c-MYB is best known as a potent oncogenic factor, its role as a master TF that stokes SMC/neointimal proliferation has also been reported (Khachigian L M. Int J Mol Sci. 2019 20). It was thus hypothesized that miR579-3p inhibited SMC proliferation and migration by downregulating c-MYB. Indeed, whereas each of the 4 cytokines upregulated c-MYB mRNA by at least 2 fold, transfection of AoSMC with miR579-3p abrogated the cytokine effect (FIG. 3C). Accordingly, miR579-3p also kept c-MYB protein at the basal level (no cytokine) as indicated by Western blotting (FIGS. 3D and 3E). Consistently, the major SMC proliferation/migration markers, including p-MEK, p-ERK, cyclin-D1, and c-MYC, were all significantly reduced by miR579-3p (FIGS. 3F-31). In accordance with previous reports in other cell types showing that these markers are c-MYB downstream target genes, herein it was observed that elevating c-MYB protein levels potently increased these 4 marker proteins (FIG. 3J). More importantly, we found that while miR579-3p transfection into AoSMCs reduced (vs moR-Con) cell proliferation and migration, overexpression of c-MYB in this miR579-3p background nearly completely reversed the miR579-3p effect (FIGS. 3K and 3L). Taken together, these results reveal miR579-3p as a novel negative regulator of c-MYB expression and its function in SMC proliferation and migration.

miR579-3p Directly Targets Two Sequences in the c-MYB 3′UTR

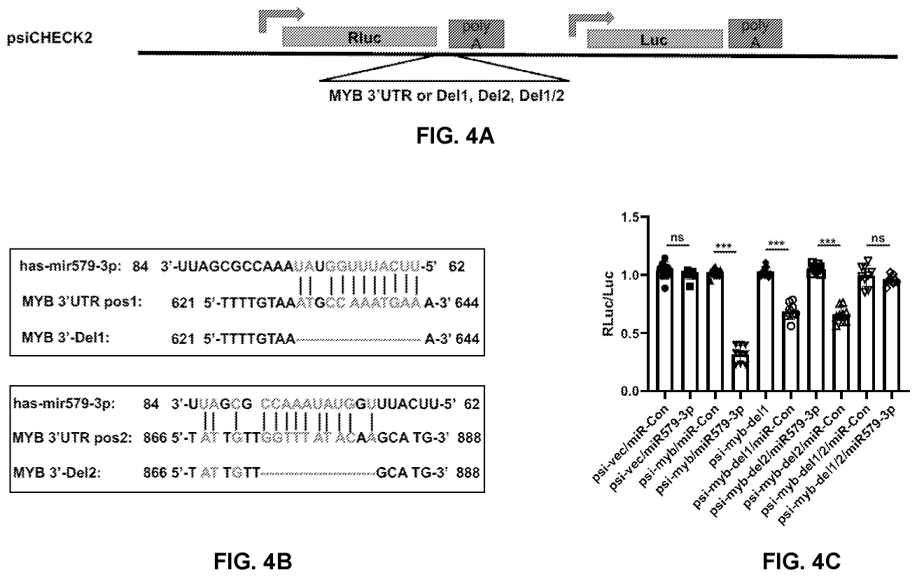

The negative regulation of c-MYB expression by miR579-3p led us to infer that c-MYB could be a direct target of miR579-3p. Indeed, via in-silico analysis, two positions were found in the 3′UTR of c-MYB mRNA that are complementary to the sequence of mature miR579-3p. Luciferase activity was then used for readout to determine how deletion of these sequences could affect miR579-3p's targeting of c-MYB mRNA. The wild type 3′UTR of c-MYB, 3′UTR with position 1 deleted, 3′ UTR with position 2 deleted, or 3′ UTR with both deletions was inserted into the downstream of the Renilla gene (FIG. 4A). The sequences of position 1 and position 2 in c-MYB 3′UTR and complementary sequences in miR579-3p are presented in FIG. 4B. As indicated in FIG. 4C, luciferase activity was remarkably reduced by miR579-3p treatment when the wild 3′UTR construct was used. However, deletion of either position-1 or position-2 partially restored, and deletion of both completely restored the luciferase activity. Thus, the luciferase assay data demonstrate that c-MYB is a direct target of miR579-3p.

miR579-3p Directly Targets a Sequence in the KLF4 3′UTR

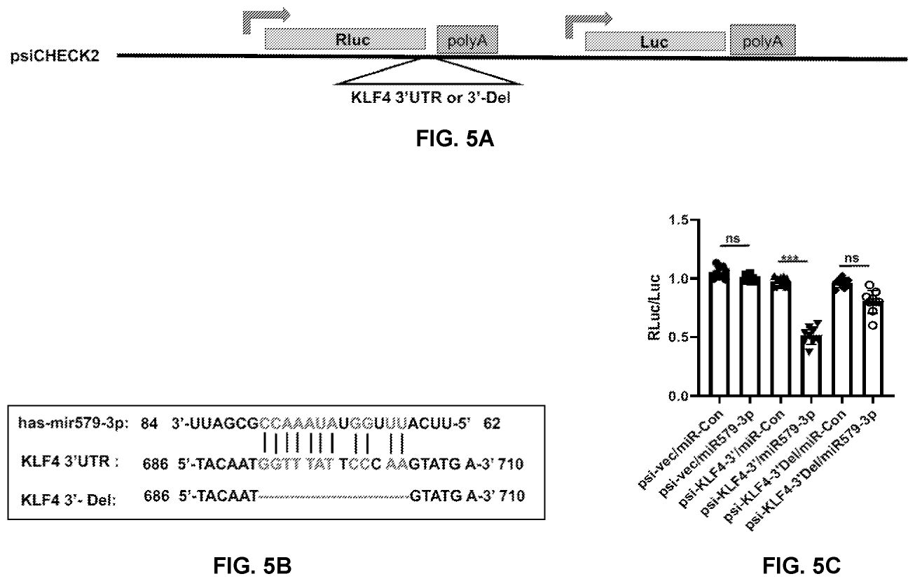

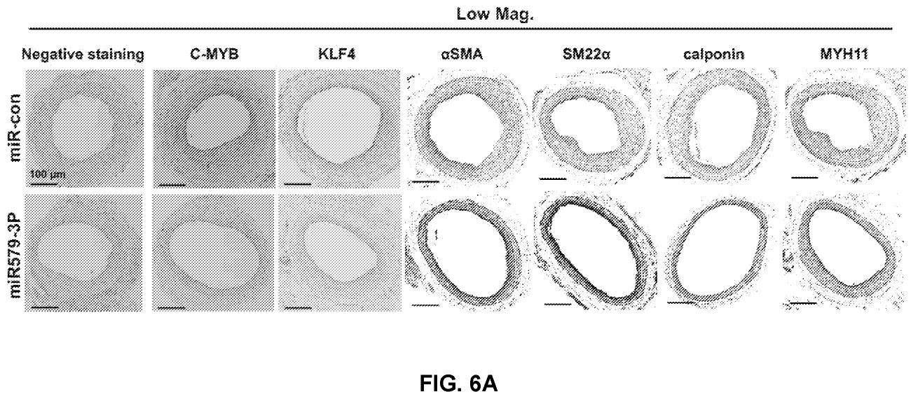

Furthermore, given that KLF4 expression was negatively regulated by miR579 (FIGS. 2D and 2E) and in-silico analysis predicted a potential miR579's target site in the KLF4 mRNA sequence, whether KLF4 could be another target of miR579-3p was examined. A luciferase assay was performed using the psiCHECK vector inserted with the wild type 3′UTR of KLF4 or the 3′UTR with a deletion of the 13 bp predicted miR579-3p target sequence (FIG. 5A). The deleted c-MYB 3′UTR sequence and the complementary sequence of miR579-3p are presented in FIG. 5B. While the luciferase activity resulting from the wild 3′UTR construct was reduced by miR579-3p treatment of AoSMCs, it was fully restored by the deletion of the predicted target sequence.

Taken together, the luciferase assays confirm that miR579-3p directly targets c-MYB and KLF4, two master TFs dictating SMC proliferation/migration and de-differentiation (Liu Y, et al. J Biol Chem. 2005 280:9719-27), in accordance with down-regulation of c-MYB and KLF4 and mitigation of AoSMC phenotypic switching caused by the treatment with miR579-3p, as observed herein in vitro.

c-MYB and KLF4 are Down-Regulated in Injured Rat Aortic Arteries Treated with Mir579-3p-Expressing Lentivirus

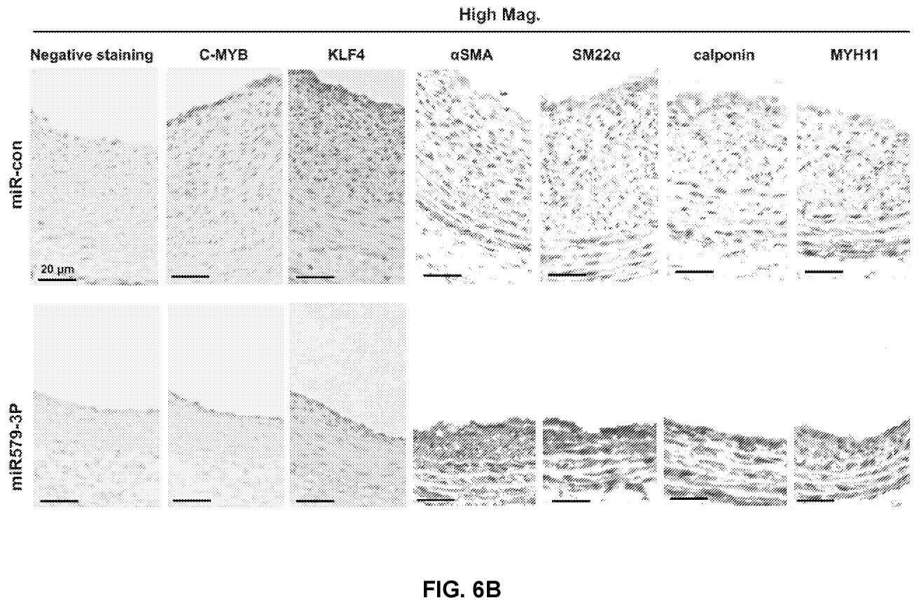

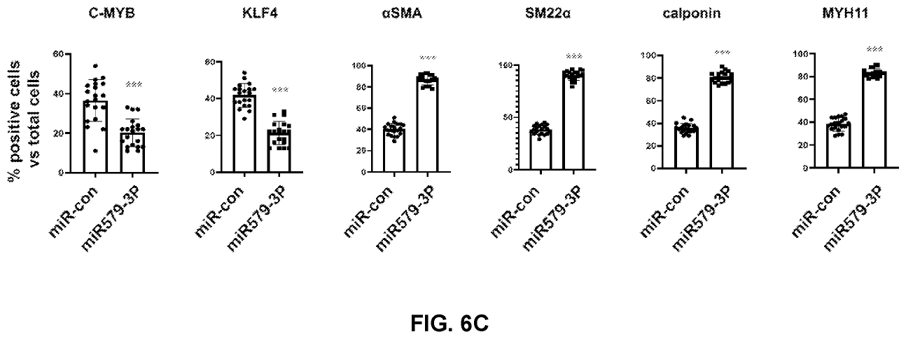

After identifying the two master TFs as novel targets of miR579-3p in AoSMCs in vitro, immunohistochemistry was next performed to confirm this novel miR579-3p-mediated regulation in vivo using cross-sections of injured arteries, which were transduced with lentivirus to express miR579-3p or miR-Con (FIGS. 6A and 6B). Consistent with the in vitro results, treatment of injured arteries with miR579-3p-expressing lentivirus markedly reduced not only c-MYB, but also KLF4 (FIG. 6C). Accordingly, the contractile proteins which are known to be negatively regulated by KLF4 (Liu Y, et al. J Biol Chem. 2005 280:9719-27) were all increased at least two-fold. Thus, these in vivo results further support the in vitro finding that miR579-3p targets the two master TFs which are critical players in SMC phenotypic switching and the development of IH (Yoshida T, et al. Circ Res. 2008 102:1548-57).

### Discussion

IH develops after reconstruction (e.g. angioplasty, bypass) of vascular vessels, leading to recurrent stenosis—a persistent medical problem despite advances in medical technologies (Inoue T, et al. JACC Cardiovasc Interv. 2011 4:1057-66), Recently, miRs have shown promise for specific targeting of IH pathogenesis. Some miRs have been reported to regulate IH (Khachigian L M. Int J Mol Sci. 2019 20; Du M, et al. iScience. 2022 25:104169; Zeng Z, et al. Circulation. 2021 143:354-371: Wang D and Atanasov A G. Int J Mol Sci. 2019 20), several being inhibitors of SMC phenotypic switching (Du M, et al. iScience. 2022 25:104169; Farina F M, et al. Circ Res. 2020 126:e120-e135; Wang Y S, et al. Cardiovasc Res. 2012 95:517-26; Yang F, et al. Circulation. 2018 137:1824-1841; Xu F, et al. Arterioscler Thromb Vasc Biol. 2015 35:2145-52). The miR145 cluster is in this IH-inhibitor category (Cheng Y, et al. Circ Res. 2009 105:158-66). However, its physiological abundance is high, limiting the therapeutic potential of further increasing its levels. miR128-3p, however, is expressed at lower levels, and is inhibitory to SMC phenotypic switching and IH (Farina F M, et al. Circ Res. 2020 126:e120-e135). Thus, previous studies encourage further investigation into miRs as potential IH-inhibitory therapeutics.

miR579-3p is shown to be a novel IH-inhibiting modulator. Consistently, increasing miR579-3p in human SMCs abrogated mitogen-stimulated SMC phenotypic switching, the principal contributor to the IH development (Lacolley P, et al. Cardiovasc Res. 2012 95:194-204; Bennett M R, et al. Circ Res. 2016 118:692-702). miR579-3p directly targeted c-MYB and KLF4, two master TFs critically involved in SMC phenotypic switching. Thus far, there are only a very small number of reports involving miR579-3p, mainly in the oncology field. While the expression of miR579-3p was suppressed in several malignant carcinomas, miR579-3p was found to inhibit cancer cell proliferation and migration (Xu J, et al. Aging (Albany NY). 2021 13:16471-16484; Barbier R H, et al. Sci Rep. 2021 11:10765; He X, et al. Am J Transl Res. 2021 13:8777-8786; Fattore L, et al. Proc Natl Acad Sci USA. 2016 113:E5005-13). This finding is in line with the result that miR579-3p inhibits human SMC proliferation and migration.

Through in-silico analysis, 3′UTR sequences were detected in KLF4 and c-MYB genes that are complementary to that of miR579-3p. Indeed, in luciferase assays, deletion of these sequences functionally confirmed their direct targeting by miR579-3p. KLF4 is known as a master TF that represses the expression of SMC contractile genes (Shankman L S, et al. Nat Med. 2015 21:628-37; Liu Y, et al. J Biol Chem. 2005 280:9719-27). In accordance, these data showed that treatment with miR579-3p in vitro or the lentivector for its expression in vivo reduced KLF4 protein and increased SMC contractile proteins. It is interesting to note that in a previous report SMG-specific KLF4 depletion in mice did not reduce but rather increased IH (Yoshida T, et al. Circ Res. 2008 102:1548-57). The exact mechanism was not clear, but possibly involved an anti-proliferative role of KLF4 (Yoshida T, et al. Circ Res. 2008 102:1548-57). This outcome from KLF4 depletion underscores that targeting a single pathway would not bring about therapeutic benefits. In this regard, miRs as candidate therapeutics are advantageous for their capacity to target multiple genes or pathways (Khachigian L M. Int J Mol Sci. 2019 20), and could thereby assume the desired potency. These data indicated that miR579-3p targeted both KLF4 and c-MYB. c-MYB is another master TF that potently promotes SMC proliferation and migration (Simons M, et al. Nature. 1992 359:67-70). As a nodal factor, c-MYB governs the expression of an array of proliferation/migration markers including MEK, ERK, c-MYC, and cyclin-D1 (Khachigian L M. Int J Mol Sci. 2019 20). However, c-MYB positively regulates SMC contractile genes (Chandy M, et al. PloS one. 2018 13:e0202778; Kolodziejska K M, et al. Circ Res. 2008 102:554-61)—a function opposite to that of KLF4 (Liu Y, et al. J Biol Chem. 2005 280:9719-27). As such, these two master TFs appear to be complementary determinants in SMC phenotypic switching. In this perspective, it would be reasonable to speculate that the net outcome from the down-regulation of both c-MYB and KLF4 could account for the decrease of SMC proliferation/migration and increase of SMC contractile proteins observed after transfection with miR579-3p. However, KLF4 may directly antagonize c-MYB's function in the activation of SMC contractile genes. In previous studies, negating c-MYB reduced injury-induced IH (You X M, et al. Circ Res. 2003 92:314-21; Simons M, et al. Nature. 1992 359:67-70), whereas SMC-specific deletion of KLF4 increased IH (Yoshida T, et al. Circ Res. 2008 102:1548-57). It is interesting to note that in this study, expressing miR579-3p in injured rat arteries reduced both c-MYB and KLF4 and attenuated IH. One explanation would be that the observed mitigation of IH was the sum of the effects of reduced c-MYB and KLF4. Alternatively, the IH-mitigating effect of miR579-3p was partially accounted for by additional targets of miR579-3p.

Although TFs such as c-MYB and KLF4 are potential interventional targets because of their importance in diseases, it has been notoriously difficult to develop small molecule drugs to selectively target these soluble proteins. To circumvent this translational barrier, miRs enable an alternative interventional approach by tuning down the expression of over-active TFs. miRs are small, easy to produce, and also amenable to modifications to enhance their beneficial properties such as stability. Moreover, various delivery platforms have been developed including nanoparticles and liposomes (Barba A A, et al. Pharmaceutics. 2019 11). In the current study, an IH-mitigating effect of miR579-3p was found via lentiviral transduction of injured arteries.

This study demonstrates that miR579-3p is an inhibitory modulator of IH and hampers the phenotypic switching of SMCs to a spectrum of aberrant behaviors—not only proliferation and migration, but also de-differentiation. This novel role of miR579-3p involves its direct targeting of c-MYB and KLF4, two master TFs in SMC pathobiology. As SMC phenotypic switching is a pathogenic process in an array of cardiovascular pathologies, including atherosclerosis, post-angioplasty restenosis, bypass graft failure, and aneurysm, research on miR579-3p should be extended beyond its herein identified role against IH.

TABLE 1
Primers used for qRT-PCR

| Target | Forward | Reverse |
|---|---|---|
| c-MYB | GGGAACAGATGGGC | GCTGGCTTTTGAAG |
| | AGAAATCG | ACTCCTGC |
| | (SEQ ID NO: 1) | (SEQ ID NO: 2) |
| GAPDH | CATGTTCGTCATGG | ATGGCATGGACTGT |
| | GTGTGAACCA | GGTCATGAGT |
| | (SEQ ID NO: 3) | (SEQ ID NO: 4) |

TABLE 2
Antibodies for Western blotting

| | | Catalog | |
|---|---|---|---|
| Antibody | Company | number | Dilution |
| c-MYB | Santa Cruz | sc-74512 | WB (1:500); IHC (1:100) |
| Phospho- | Cell signaling | 9121 | WB (1:1000) |
| MEK | technology | | |
| Phospho- | Cell signaling | 4370 | WB (1:2000) |
| ERK | technology | | |
| Cyclin D1 | Cell signaling | 55506 | WB (1:1000) |
| | technology | | |
| c-Myc | Cell signaling | 13987 | WB (1:1000) |
| | technology | | |
| GAPDH | Cell signaling | 2118 | WB (1:3000) |
| | technology | | |
| KLF4 | Proteintech | 11880-1-AP | WB(1:1000); IHC(1:200); |
| | | | IF(1:100) |
| SMA | Proteintech | 14395-1-AP | WB(1:5000); IHC(1:3000); |
| | | | IF(1:300) |
| SM22 | Proteintech | 10493-1-AP | WB(1:1000); IHC(1:100); |
| | | | IF(1:200) |
| Calponin | Proteintech | 13938-1-AP | WB(1:1000); IHC(1:100); |
| | | | IF(1:100) |
| MYH11 | Proteintech | 21404-1-AP | WB(1:2000); IHC(1:2000); |
| | | | IF(1:200) |

TABLE 3
Primers construction of c-MYB 3′UTR and KLF4 3′UTR in vector.

| Cloned | | |
|---|---|---|
| region | Forward | Reverse |
| c-MYB 3′UTR | GACTCATACAGCTGGCACTCGAGGGACGCTGGTCATG | GACTCATACAGCGGCCGCAAAGTGCCTTGAGTCTGCT |
| Wild type | TGAGAC (SEQ ID NO: 5) | CC (SEQ ID NO: 6) |
| c-MYB 3′UTR | TTTTATTCAGTAATTTAATTTTGTAAAAACGTTTTTT | TAAGACCATAGCAGCAAAAAACGTTTTTACAAAATTA |
| with Del1 | GCTGCTATGGTCTTA (SEQ ID NO: 7) | AATTACTGAATAAAA (SEQ ID NO: 8) |
| c-MYB 3′UTR | TATTGTGGTTTTTTTGTTATTGTTGCATGCGTTGCAC | CCAAAAAAGAAGTGCAACGCATGCAACAATAACAAAA |
| with Del2 | TTCTTTTTTGG (SEQ ID NO: 9) | AACCACAATA (SEQ ID NO: 10) |
| KLF4 3′UTR | ACTCATACAGCTGGCACTCGAGATCCCAGACAGTGGA | ACTCATACAGCGGCCGCATTCTCACCTTGAGAATGCA |
| Wild type | TATGAC (SEQ ID NO: 11) | (SEQ ID NO: 12) |
| KLF4 3′UTR | CAGATGTGCAATAATTTGTACAATGTATGCCTTAAGC | CACATTTGTTCTGCTTAAGGCATACATTGTACAAATT |
| with Del | AGAACAAATGTG (SEQ ID NO: 13) | ATTGCACATCTG (SEQ ID NO: 14) |

### Example 2

### Material and Methods

### Materials

Collagen IV targeting peptide was purchased from GenScript. HL-60 (CCL-240) cells and Iscove's Modified Dulbecco's Medium (IMDM, 30-2005) were from ATCC. AF647 labeled scramble RNA was synthesized from ExoNanoRNA (Columbus, OH). Cholesterol-PEG1000-Maleimide (3024-1000) was from Nanosoft Biotechnology LLC. MLV5 (890000) (N1-[2-((1S)-1-[(3-aminopropyl)amino]-4-[di(3-amino-propyl)amino]butylcarboxamido)ethyl]-3,4-di[oleyloxy]-benzamide), Extruder set, PC membranes (serial pore size: 610005, 610006, 610007, 610009, 610010), and filter support (610014) were purchased from Avanti.

### Neutrophil Cells Culture and Induction

HL-60 cells were cultured with IMDM containing 20% FBS in a 37° C. incubator (5% CO₂). The medium was replaced every 2-3 days. Cells were induced with 1.5% DMSO for 6 days to form neutrophil cells (Millius A., et al. Methods Mol. Biol. 2010 591:147-158; Tarella C., et al. Cancer Res. 1982 42(2):445-449). Induced cells were collected using a centrifuge of 800 g for 10 min. The cell pellets were kept in −80° C.

### Neutrophil Cell Membrane Extraction

The membrane was extracted following the references with some modifications (Zhang, Q., et al. Nature Nanotech 2018 13:1182-1190). In brief, frozen neutrophil cells were thawed and washed with 1×PBS three times (centrifugation at 800 g). Cells were then suspended in a hypotonic lysing buffer containing 30 mM Tris-HCl (pH 7.5), 225 mM D-mannitol, 75 mM sucrose, 0.2 mM EGTA, and a protease and phosphatase inhibitor cocktail. Cells were then disrupted using a Dounce homogenizer with a tight-fitting pestle (>20 passes). The homogenized solution was centrifuged at 20,000 g for 25 min at 4° C. The pellet was discarded and the supernatant was centrifuged again at 100,000 g for 35 min at 4° C. Following the centrifugation, membranes were collected as the pellet and washed twice with 0.2 mM EDTA in water.

The alternative method is using a hypoosmotic procedure (Sloan, E. P., et al. J Exp Med 1981 153(5):1316-28). In brief, the cells were dissolved in 40 mM sucrose for 5 min. The suspension was then homogenized 20 times in a Dounce homogenizer with a tight-fitting pestle A. Isoosmolarity was restored by the addition of an appropriate volume of chilled 2 M sucrose. Centrifugation at 800 g for 5 min separated the cell homogenate into a crude nuclear pellet and a partial extract. The above homogenization was repeated on the crude pellet with isotonicity being restored this time through the addition of 1.45 M NaCl. After repeating the 800 g, 5-min spin, both supernatants were combined. The supernatants were centrifuged at 100,000 g for 35 min at 4° C. The pellet was suspended in 75 mM NaCl and 125 mM sucrose.

### The Reaction of Maleimide and Sulfhydryl Groups

The maleimide group reacts specifically with sulfhydryl groups when the pH of the reaction mixture is between 6.5 and 7.5 (Martinez-Jothar, L., et al. Journal of Controlled release. 2018 282:101-109). The peptide was dissolved in solvent DMF (10 mM) and Cholesterol-PEG-Maleimide was dissolved in water (10 Mm). Then Cholesterol-PEG-Maleimide and peptide were mixed at a 1:1 ratio at room temperature for 3 hours. The compound of Cholesterol-PEG-Maleimide-Peptide was named C-P-M-P here.

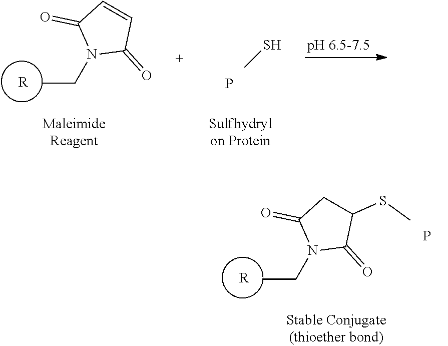

### MLV5/RNA Core Preparation

MLV5 2 mg was dissolved in 5 μl chloroform, then add water to form 1 mM stock. MLV5/RNA complexes were prepared by adding MLV5 solution dropwise to AF647 labeled scramble RNA solution to an equal volume (2:1 ratio of mol). Then quickly mix using vortex before incubating them at room temperature for 30 min to form complexes (Leal C., et al. Langmuir. 2011 27(12):7691-7; Bouxsein N F., et al. Biochemistry. 2007 46(16):4785-92). The complex core was named M-R here.

### Formation of Nanoparticle

M-R, extracted neutrophil membrane, and C-P-M-P were mixed and incubated in ice for 30 min before passing through the serial pore size of PC membranes (from 1000 nm, 800 nm, 400 nm, 200 nm, then 100 nm) using an extruder. The nanoparticle solution buffer was changed to PBS using dialysis. The nanoparticle solution was then subjected to condense using a vacuum centrifuge till the appropriate volume.

Cholesterol-PEG-MAL: Cholesterol-poly(ethylene glycol) with conjugated maleimide is a lipophilic lipid PEG conjugate.

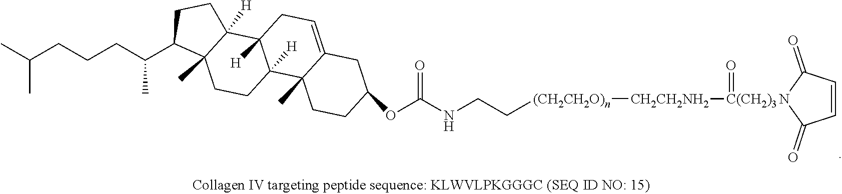

### Example 3. Biomimetic Torpedo for Stent-Free and Targeted Gene Therapy to Prevent Restenosis

Non-viral gene therapy with small RNAs has come of age. After early setbacks, the past two years have been marked by 3 consecutive FDA approvals of siRNA therapy. However, there remains the lack of an approved siRNA or miRNA therapy for cardiovascular disease because of several major hurdles. For example, naked siRNAs or miRNAs delivered in the circulation do not aggregate at the site of the lesion in the vessel wall because they are sequestered by the major organs including the liver. Moreover, siRNAs/miRNAs can be immunogenic and rapidly destroyed by the internal immune system. We have engineered a unique biomimetic torpedo which can circumvent these major issues. That is, siRNA/miRNA is harbored inside the torpedo shell made of neutrophil membranes in hybrid with a liposome membrane, which enables lesion-targeting and shielding from immunogenicity. Surprisingly, biomimetic torpedoes are only minimally detected or sequestered in major mouse organs including the liver, heart, kidney, and spleen. This is a detriment of other biomimetic approaches—the particles are sequestered in the major organs and do not make their way to the site of arterial injury. In preclinical and clinical studies, a general problem is that intravenously delivered therapeutic agents are mostly trapped in major organs, whether they are delivered in liposomes or polymer nanoparticles or biomembrane-camouflaged particles. Moreover, the compatible payloads of the biomimetic torpedo can be any nucleotide agents, whether siRNA, miRNA, aptamers or mRNA. Therefore, a biomimetic torpedo that has a neutrophil/liposome hybrid membrane coating would be a transformative contribution to broad translational endeavors and deserves supports for further development.

Disclosed herein a highly innovative “biomimetic torpedo” system for stent-free and non-thrombogenic targeted therapy to prevent restenosis—a paradigm radically different from the status quo which is drug-eluting stents that predispose patients to in-stent restenosis and thrombosis.

Angioplasty is commonly used to reopen occluded arteries. To prevent restenosis, drug-eluting stents are implanted, but this paradoxically promotes in-stent restenosis and thrombosis. The angioplasty/stenting procedures overstretch the artery and tear the endothelial inner lining, hence inevitably incurring open wounds on the inner vessel wall, where neointimal scars and/or thrombi form leading to restenosis.

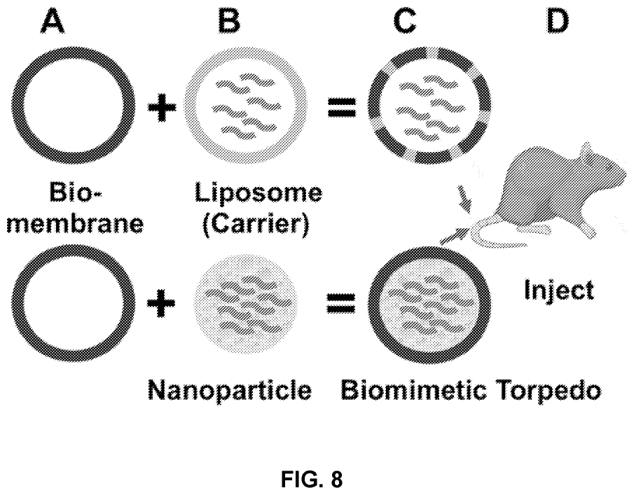

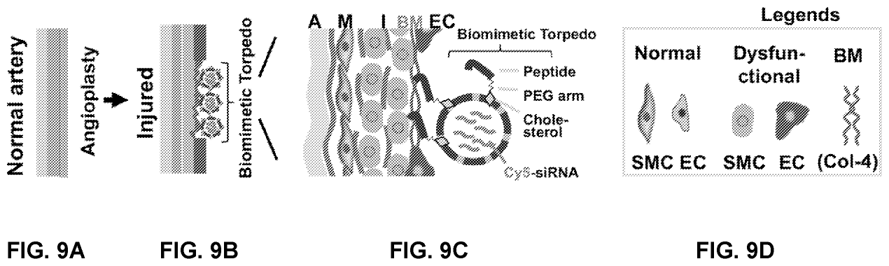

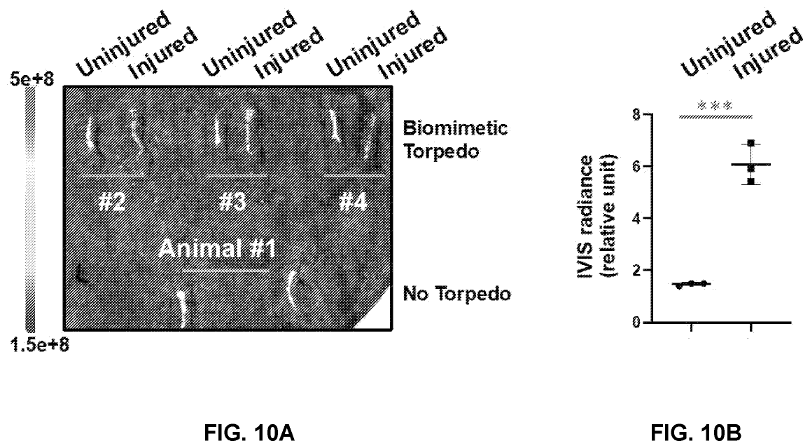

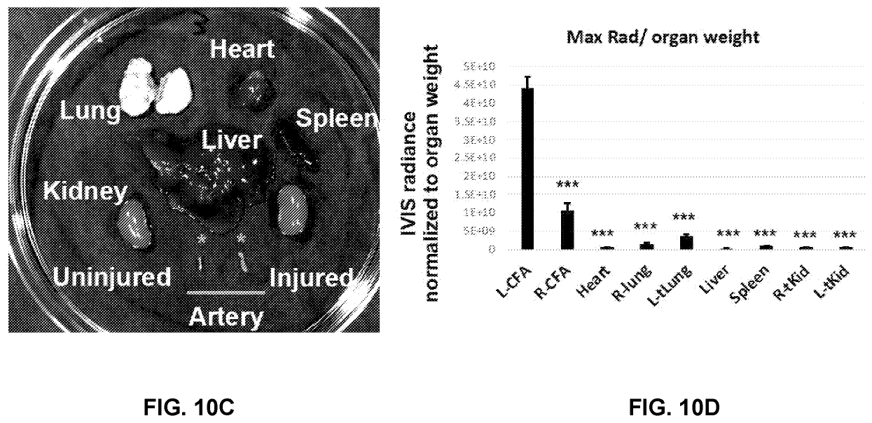

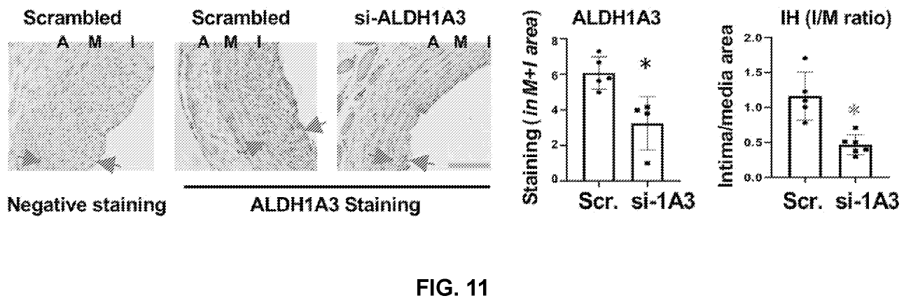

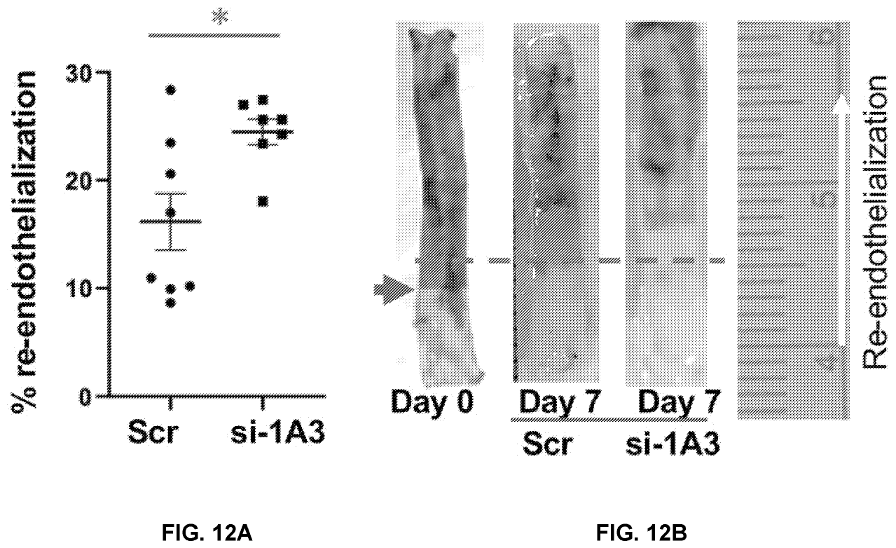

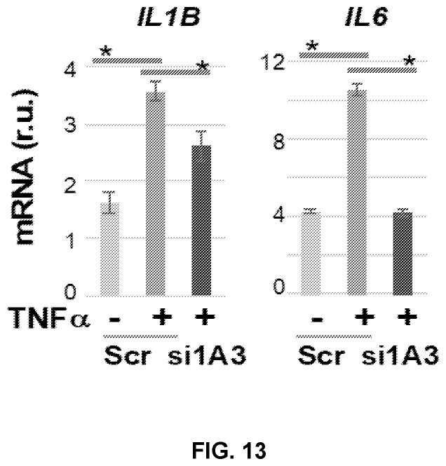

Despite innumerable studies, recurrent stenotic disease persists. To address this problem, an elegant stent-free (injectable) biomimetic torpedo system was developed (FIG. 8). Neutrophils are well known as first responders to wounds or sites of injury. However, their superb wound-honing ability has not been translated into vascular treatments. To formulate a biomimetic torpedo, we first disrupt neutrophils to destroy the intracellular machinery that may cause unwanted effects, and only use the cell membrane to retain its wound-honing property. An extruder was then use to produce neutrophil membrane capsules which are further decorated with a short peptide. This peptide specifically targets collagen-IV, the most abundant protein in the basal membrane which is ex-posed due to endothelium damage after angioplasty (FIG. 9). The capsules, herein dubbed as biomimetic torpedoes, are capable of double targeting. That is, while intravenously injected torpedoes are guided by their neutrophil membrane to the site of angioplasty, the peptides plugged in the membrane specifically bind collagen-IV that is exposed by the injury (FIG. 10 shows targeted siRNA delivery to injured artery but not uninjured artery and no accumulation in major organs). Thereby, these torpedoes provide an ideal vehicle to deliver targeted gene therapy; ALDH1A3-specific siRNA can be used as the torpedo payload. ALDH1A3-specific siRNA delivered to the injured rat artery wall mitigated restenosis (herein measured as neointimal hyperplasia or IH, FIG. 11). Dysfunction of two vital cell types (smooth muscle and endothelial cells) in the vessel wall critically contributes to neointimal scar formation and restenosis. Current drug-eluting stents attenuate smooth muscle dysfunction but not endothelial dysfunction; rather, these drugs often harm the endothelium exacerbating thrombosis. A master target was identified, ALDH1A3 (PMID: 31513972)—silencing it with siRNA blocks not only smooth muscle cell dysfunction (hence reduced IH, FIG. 11) and endothelial dysfunction. Indeed, ALDH1A3-specific siRNA delivered to the injured rat artery wall not only reduced IH and restenosis but also preserved endothelial cell (EC) function, as indicated by enhanced re-growth of injured endothelium in vivo (i.e. re-endothelialization, see FIG. 12) and inhibited pro-inflammatory cytokine expression in cultured ECs (FIG. 13). Whereas ALDH1A3 was traditionally known as an enzyme, recent studies (Circulation, 2021. PMID: 33764154) have shown that ALDH1A3 promotes histone acetylation (via acetyl-CoA production) eliciting epigenetic remodeling, thereby exerting profound control of multiple downstream pathways. In this light, though seemingly a single agent, ALDH1A3-specific siRNA loaded in biomimetic torpedoes confers powerful ammunition to affect a large number of pathogenic path-ways involved in restenosis disease. In addition to ALDH1A3-targeting siRNA, we could also use miR579 as a torpedo payload. miR579-3p was identified as a novel small-RNA inhibitor of restenosis. Because of similar physical properties such as negative charges, either siRNAs or miRNAs can be readily loaded into the biomimetic torpedo by using liposomes. Therefore, the final product of the biomimetic torpedo has a shell made of neutrophil membranes in hybrid with liposome membranes. This unique hybrid membrane may have enabled the injected torpedo to evade entrapment in major organs in the tests using mice (FIG. 9).

In summary, a biomimetic torpedo with a shell of neutrophil/liposome hybrid membranes is provided for injectable and lesion-targeted gene therapy delivery. This system may represent a significant advance in cardiovascular drug deliver

### Example 4. A Novel Role for ALDH1A3-Knockdown in Preserving EC Function and Mitigation of IH

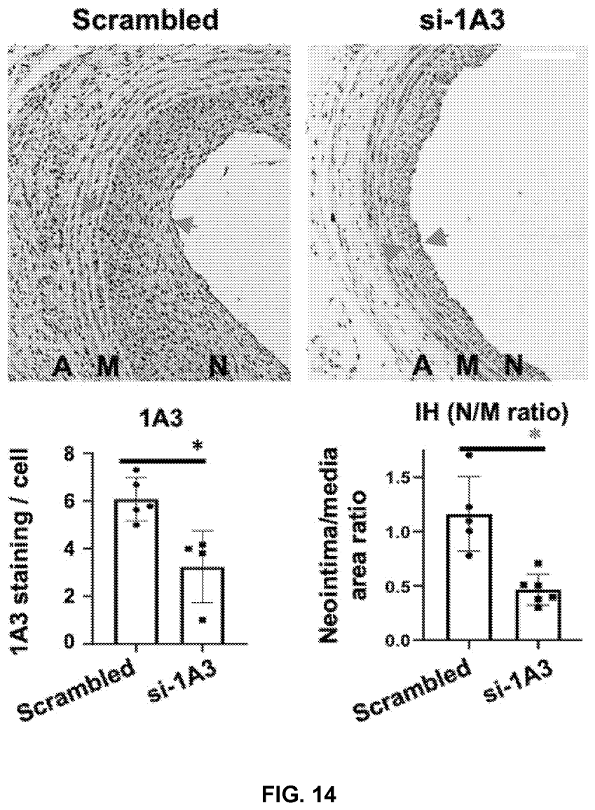

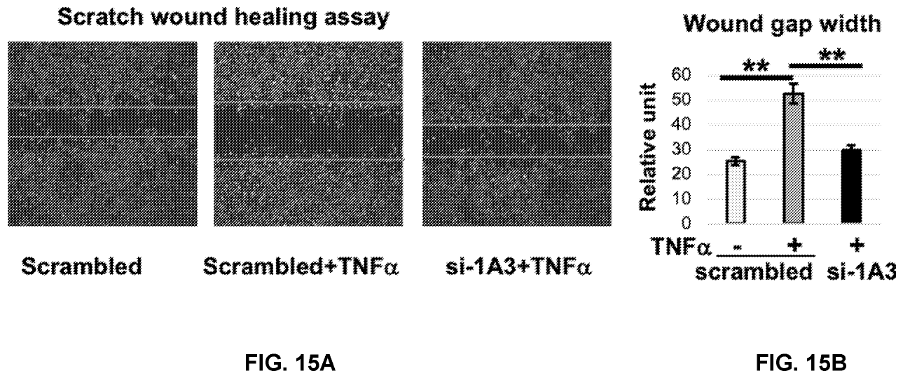

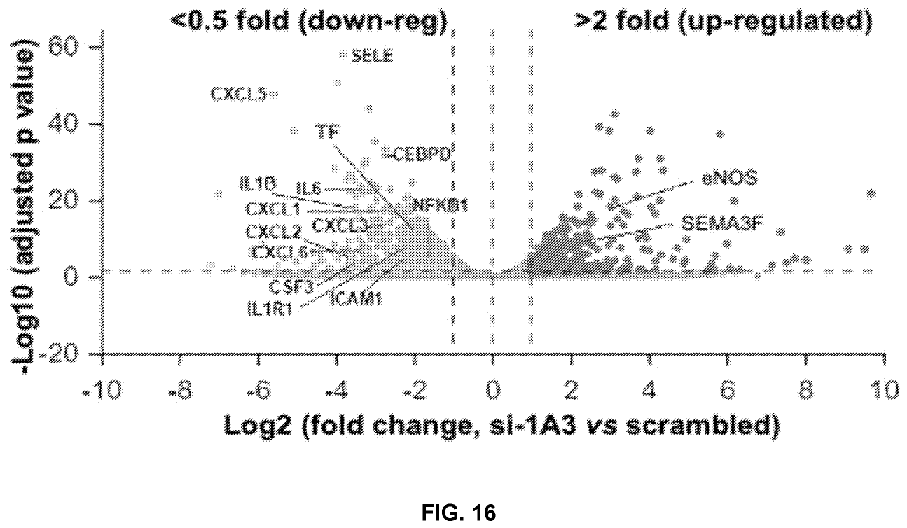

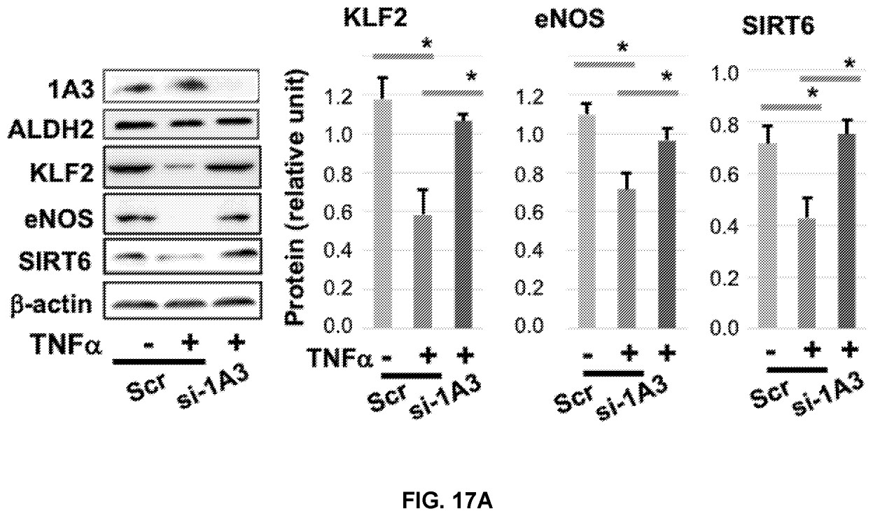

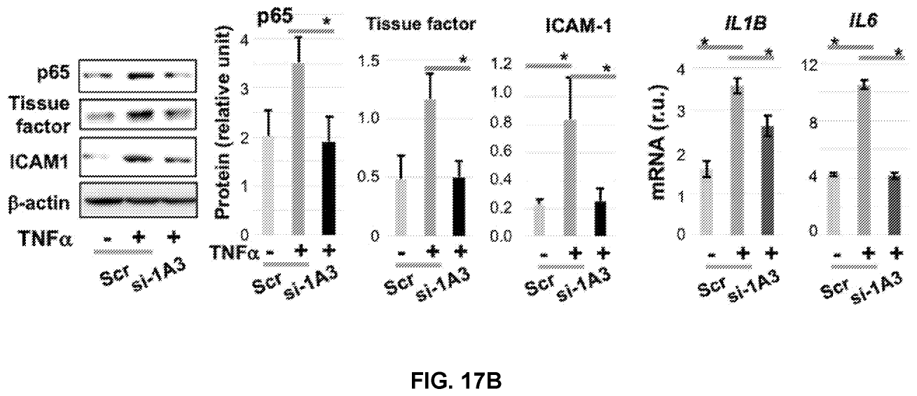

ALDH1A3(1A3)-specific knockdown in angioplasty-injured rat arteries preserved EC function, accelerated re-endothelialization, FIGS. 12A and 12B) and mitigated IH (FIG. 14). Moreover, siRNA-1A3 knockdown enhanced the recovery of ECs after scratch wound in culture dish that mimics re-endothelialization (FIGS. 15A and 15B). Transcriptomics and biochemical analysis shows that 1A3-siRNA increased eNOS, a key EC-protective factor, and inhibited pro-inflammatory cytokine expression (FIG. 16). Importantly, 1A3-siRNA increased KLF2 protein (FIG. 17)—the well-established EC-protective master transcription factor (Lin Z, et al. Circ Res. 2005 96:e48-57) that directly activates eNOS transcription (SenBanerjee S, et al. J Exp Med. 2004; 199:1305-15) and at the same time represses pro-inflammatory cytokines and tissue factor (Lin Z, et al. Circ Res. 2005 96:e48-57; Dekker R J, et al. Blood. 2006; 107:4354-63; Lin Z, et al. Arterioscler Thromb Vasc Biol. 2006; 26:1185-9; Li W, et al. Aging. 2021; 13:12996-13005).

Overall, these studies support the premise that elevated 1A3 expression drives the dysfunction of both ECs and SMCs associated with IH development; molecular targeting of 1A3 has the potential to improve EC function and thus safely and effectively mitigate IH.

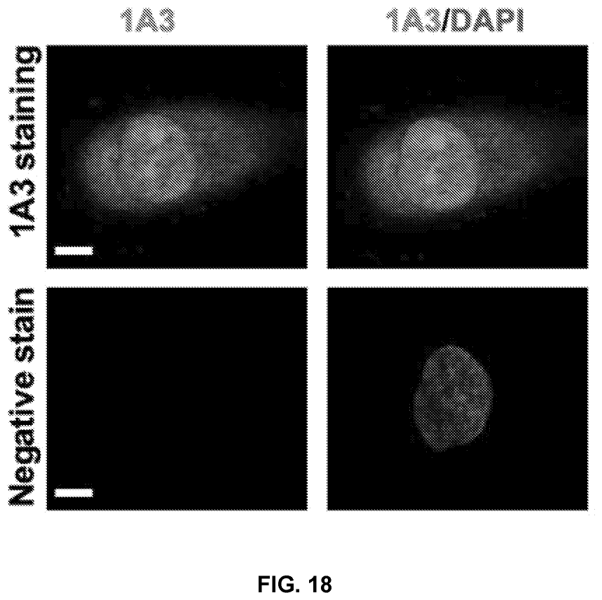

As disclosed herein, knockdown of 1A3 leads to enhancement of KLF2 (FIG. 17). While 1A3 is commonly known as a cytosol-localized metabolic coordinator, new data showed that 1A3 al-so occurs in the EC nucleus (FIG. 18). Nullifying 1A3 conferred dual benefits of endothelial protection and IH mitigation—a strategy to tackle the bipartite etiology of EC dysfunction and SMC hyper-proliferation.

A major challenge for siRNA gene therapy is to deliver it to the right place (Pi F, et al. Nat Nanotechnol. 2018; 13:82-89). A Biomimetic Torpedo (FIG. 8) was created for injectable siRNA delivery that can target the wounded arterial wall without entrapment in major vital organs. As such, this injectable (stent-free) delivery paradigm differs radically from the status quo, DES, which has unwanted side effects on re-endothelialization and exacerbates thrombosis (Otsuka F, et al. Nat Rev Cardiol. 2012; 9:439-53; Byrne R A, et al. Eur Heart J. 2015; 36:3320-31; Steffel J, et al. Circulation. 2005; 112:2002-11).

Moreover, the Biomimetic Torpedo can be repeatedly injected, conferring adjustable regimens towards personalized precision medicine.

Ultimately, the combined innovations could lead to stent-free, EC-protective (non-thrombogenic), siRNA-based targeted therapy for safe and effective management of IH.

### Example 5. Understand the Physiologic Role for ALDH1A3 in Driving EC Dysfunction Associated with IH, and Eluci-Date the Mechanisms Underlying ALDH1A3's Role in Controlling the p65-KLF2 (eNOS or Inflammation) Axis in ECs

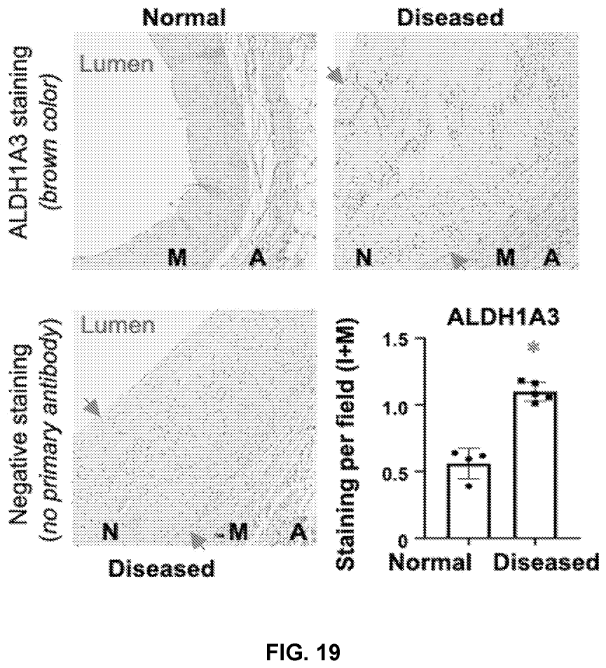

There was increased immunostaining of 1A3 in human coronary arteries from patients of IH (FIG. 19), linking 1A3 to human IH disease. Studies revealed that 1A3 knockdown is endothelial-protective. In angioplasty-wounded rat arteries, local infusion of lentivirus to express 1A3-specific shRNA increased re-endothelialization by ˜1.5 fold (from 16.5% in scrambled controls to 25.0% in the si-1A3 treatment group) (FIGS. 12A and 12B) and reduced IH by 66.6% (FIG. 14). Furthermore, 1A3-siRNA treatment (compared to Scr control) accelerated the recovery of scratch-wounded EC culture that mimics re-endothelialization (FIGS. 15A and 15B).

Mechanistically, as revealed by transcriptomic analysis using ECs, 1A3-siRNA elevated the expression of eNOS which maintains EC phenotype and growth, and also reduced the expression of EC-detrimental genes including pro-inflammatory cytokines (e.g. IL-1B, IL-6, CXCL1, 2, 5, 6, ICAM1, CSF3), E-selectin (SELE), and tissue factor (TF) (FIGS. 16A and 16B) which promote IH and thrombosis. These findings were also supported by immunoblotting and qRT-PCR (FIG. 17).

Importantly, 1A3 knockdown increased KLF2 protein, a known EC-protective master transcription factor which activates the transcription of eNOS and other EC-protective factors such as SEMA3F and SIRT6, and together leads to repression of the aforementioned EC-detrimental genes. The specificity of the siRNA was confirmed by reduced 1A3 protein but not ALDH2 (FIG. 16A) nor other ALDH isoforms (RNA-seq data).

1A3 also exists in the EC nucleus (FIG. 17) whereas it is traditionally deemed as a cytosolic enzyme (Duan J J, et al. Int J Cancer. 2016; 139:965-75). These data support our hypothesis that 1A3 negatively regulates KLF2 levels in ECs through a mechanism whereby the 1A3 enables p65 protein acetylation hence its resistance to proteolysis, leading to re-pressed KLF2 expression and hence EC dysfunction.

### Example 6. Establish Wound-Targeting Delivery of siRNA Therapy for Endothelium-Protective Mitigation of IH

Drug-eluting stents (DES) are a major advance in IH (or restenosis) management but accompanied by EC-toxic, thrombogenic side effects (Jukema J W, et al. Nat Rev Cardiol. 2012 9:79-90; Byrne R A, et al. Eur Heart J. 2015; 36:3320-31; Inoue T, et al. JACC Cardiovasc Interv. 2011; 4:1057-66). It was envisioned that an endothelium-protective, stent-free therapy can circumvent these problems and will arise only with synergistic innovations in both therapeutic targets and delivery methods. To this end, it was found that 1A3 is a promising target for endothelium-protective intervention, and 1A3-specific siRNAs would provide desired therapeutics, especially considering that 1A3 is the only isoform that is prominently upregulated in IH conditions (Xie X, et al. iScience. 2019 19:872-882; Li D, et al. Circulation. 2021 143:2074-2090; Puttini S, et al. Front Cardiovasc Med. 2018 5:90). However, lesion-homing delivery of siRNA needs to be solved for endovascular treatment of IH. Numerous delivery methods exist for siRNA gene therapy, including liposomes and polymer-based nanoparticles (Xia Y, et al. Biomaterials. 2016 79:56-68; van der Meel R, et al. J Control Release. 2014 195:72-85). There are FDA-approved liposome products (e.g. SNALP77 and DOTAP) (Barba A A, et al. Pharmaceutics. 2019 11; Bulbake U, et al. Pharmaceutics. 2017 9).

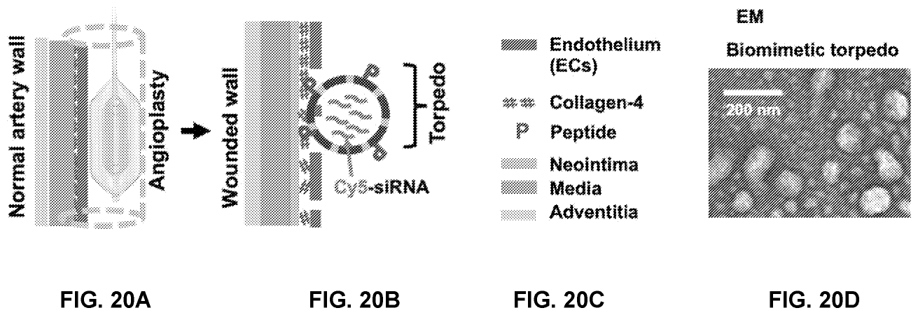

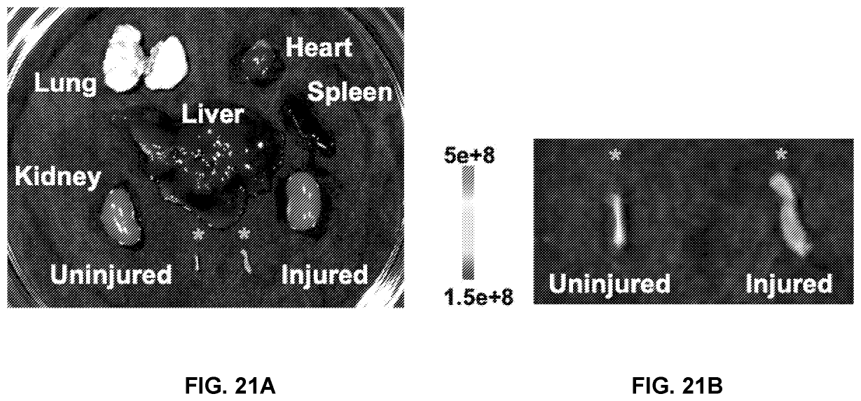

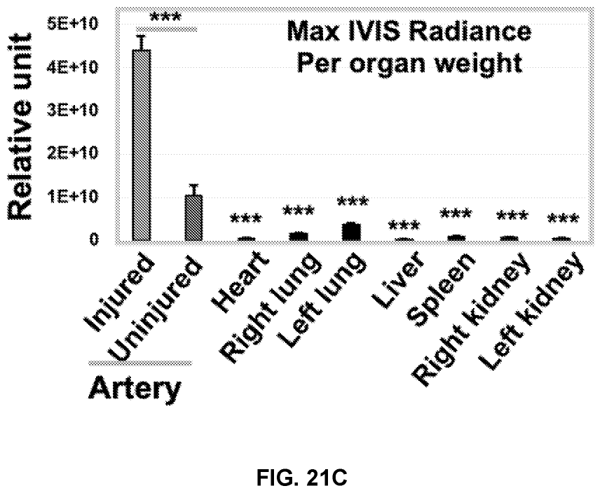

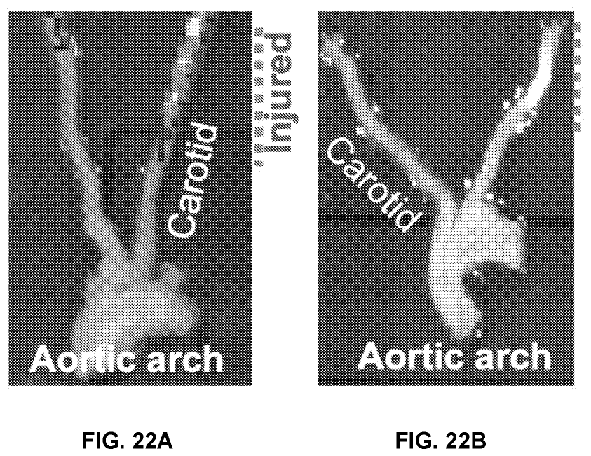

Unfortunately, these siRNA carriers bear a common shortcoming, i.e. inability to target the IH-prone and thrombogenic wounds on the arterial wall inflicted by angioplasty/stenting. In contrast, the disclosed Biomimetic Torpedo (FIG. 20) with a “shell” of neutrophil- or exosome-derived biomembrane showed robust homing to arterial wounds in either mouse (FIG. 21) or rat arteries (FIGS. 22A and 22B).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A biomimetic targeting system, comprising a neutrophil cell membrane capsule that encapsulates a therapeutic RNA.

2. The system of claim 1, further comprising a plurality of peptides that specifically target collagen-IV inserted into the neutrophil cell membrane capsule.

3. The system of claim 2, wherein the peptide comprises the amino acid sequence SEQ ID NO:15.

4. The system of claim 2, wherein the peptide is conjugated to a cholesterol molecule for integration into the neutrophil cell membrane.

5. The system of claim 1, wherein the therapeutic RNA is an siRNA, shRNA, mRNA, or miRNA.

6. The system of claim 5, wherein the therapeutic RNA comprises miR579-3p.

7. The system of claim 6, wherein the therapeutic RNA comprises the nucleic acid sequence UUCAUUUGGUAUAAACCGCGAUU (SEQ ID NO:16).

8. The system of claim 5, wherein the therapeutic RNA is an ALDH1A3-specific siRNA.

9. The system of claim 1, wherein the neutrophil cell membrane capsule further comprises liposome lipids.

10. The system of claim 9, wherein the capsule is produced by a process comprising fusing a liposome encapsulating the therapeutic RNA with a neutrophil biomembrane.

11. The system of claim 1, wherein the neutrophil cell membrane capsule encapsulates a polymeric nanoparticle loaded with the RNA therapeutic.

12. A method for preventing restenosis in a subject, comprising administering to the subject an effective amount of the targeted gene therapy system of claim 1.

13. The method of claim 12, wherein the targeted gene therapy system is administered within 1 day of an angioplasty treatment.

14. The method of claim 13, wherein the subject is not given a stent.
